(19) United States
(12) Patent Application Publication
(10) Pub. No.: US 2026/0007899 A1
(43) Pub. Date: Jan. 8, 2026

(54) ELECTRODE ASSEMBLY CONFIGURATIONS FOR ENDOVASCULAR THERAPY DEVICE

(71) Applicant: Covidien LP, Mansfield, MA (US)

(72) Inventors: Robert L. Olson, White Bear Lake, MN (US); Dale F. Seeley, Spring Park, MN (US)

(21) Appl. No.: 19/253,032

(22) Filed: Jun. 27, 2025

Related U.S. Application Data
(60) Provisional application No. 63/667,392, filed on Jul. 3, 2024.

(51) Int. Cl. A61N 1/375; A61N 1/05

(52) U.S. Cl. CPC A61N 1/37516; A61N 1/05; A61N 1/37518

(57) ABSTRACT

In some examples, an endovascular medical device system includes an elongated body configured to be introduced in a blood vessel of a patient. The system includes an expandable structure at a distal portion of the elongated body. The expandable structure includes an expandable body portion including a plurality of interconnected struts and a plurality of electrode attachment elements. The system further includes one or more electrode assemblies mechanically coupled to the expandable structure via the electrode attachment elements. Each electrode assembly includes an electrically conductive portion and an electrically insulative portion. The electrically insulative portion of each respective electrode assembly is configured to facilitate mechanical coupling of the respective electrode assembly to the expandable structure. In some examples, the electrode attachment elements of the expandable structure are configured to orient respective electrode assemblies such that the electrically conductive portion of each electrode assembly faces radially outward from the expandable structure.

### CROSS-REFERENCE TO RELATED APPLICATIONS

This application claims the benefit of and priority to U.S. Provisional Patent Application Ser. No. 63/667,392 filed Jul. 3, 2024, the entire disclosure of which is incorporated by reference herein.

### TECHNICAL FIELD

This disclosure relates to electrical stimulation therapy.

### BACKGROUND

Medical devices, such as electrical stimulation devices, may be used in different therapeutic applications, such as vagus nerve stimulation (VNS) and/or deep brain stimulation (DBS). A medical device may be used to deliver therapy to a patient to treat a variety of symptoms or patient conditions. In some therapy systems, an external or an implantable electrical stimulator delivers electrical stimulation therapy to a target tissue site within a patient with the aid of one or more electrode assemblies and/or senses one or more patient parameters with the aid of the one or more electrode assemblies.

### SUMMARY

This disclosure describes example endovascular medical devices and systems configured to endovascularly deliver electrical stimulation therapy to a patient (e.g., to one or more nerves or brain targets) and/or sense one or more patient parameters (e.g., nerve signals, brain signals, and/or other physiological parameters), and related methods. In particular, this disclosure describes configurations for electrode assemblies of endovascular medical devices and systems that facilitate delivery of electrical stimulation therapy and/or sensing patient parameters from an endovascular location.

In the examples described herein, an endovascular therapy system includes one or more electrode assemblies that are carried by an expandable structure at a distal portion of an elongated body (e.g., a medical lead). The expandable structure is configured to transform between a delivery (e.g., compressed or relatively low-profile) configuration and a deployed (e.g., expanded) configuration. The expandable structure (e.g., a stent, or stent-like structure) includes struts and a plurality of electrode attachment elements. The electrode attachment elements facilitate mechanical coupling of the electrode assemblies to the expandable structure. Thus, because electrode assemblies can be fabricated separately from the expandable structure, properties of electrode assemblies can be selected and/or adjusted during fabrication, manufacturing, and/or assembly for a particular end use, and the electrode assemblies can subsequently be attached to the expandable structure via the electrode attachment elements.

Each electrode attachment element, which may include or be affixed to a portion of one or more struts of the expandable structure, includes one or more structural features (e.g., projections) configured to facilitate mechanical coupling of electrode assemblies to the expandable structure. Each electrode assembly is configured to receive one or more of the structural features (e.g., projections) of respective electrode attachment elements to mechanically couple the respective electrode assemblies to the expandable structure. The electrode attachment elements described herein can be configured to minimize or even prevent rotation of the electrode assemblies with respect to the struts of the expandable structure.

Minimizing, or even preventing, rotation of electrode assemblies with respect to struts can facilitate directional orientation of electrode assemblies with respect to the expandable structure. For example, by minimizing, or even preventing, rotation of electrode assemblies with respective to struts, an electrically conductive portion of each electrode assembly can be configured to face radially outward (relative to a central longitudinal axis of the expandable structure) from the expandable structure with little or no possibility of the electrically conductive face being oriented radially inward, such as away from a blood vessel wall. Said another way, the system can bias transmissions of electrical signals to and/or sensing of signals from tissue surrounding blood vessel as compared to radially inward from the blood vessel wall. In this way, the system is configured to facilitate directional electrical stimulation therapy and/or directional sensing, such as in a direction radially outward from the expandable structure and/or a blood vessel and towards a blood vessel wall. By fixing the particular surface of the electrode that faces radially outward towards a vessel wall, the systems described in this disclosure may be relatively more efficient (e.g., in terms of power used and/or power lost) during electrical stimulation therapy and/or sensing, such as compared to other types of systems that do not fix or orient conductive surfaces in a particular direction. Further, the directional electrical stimulation facilitated by the electrode assemblies described herein can help direct electrical stimulation signals to a specific target tissue site to enhance therapy efficacy and reduce possible adverse side effects from stimulating unintended tissue sites (e.g., particular nerves or brain targets).

In some examples herein, electrode assemblies include structural features for mechanically coupling to expandable structures (e.g., stents or stent-like structures). In some examples, the structural features that facilitate mechanical coupling of the electrode assemblies to the expandable structures are also configured to electrically insulate a given electrode assembly from the expandable structure. In other words, structural features of the electrode assemblies described herein can facilitate mechanical coupling of the electrode assemblies to the expandable structure as well as facilitate the secondary purpose of electrically insulating the electrode assemblies from the expandable structure. This dual purpose of facilitating mechanical coupling and facilitating electrical insulation can reduce or even eliminate extra material that would otherwise need to be incorporated into the electrode assemblies to facilitate each of the mechanical coupling and electrical insulation functions. Reducing or even eliminating the extra material can help maintain a relatively low profile medical device suitable for endovascular delivery to a target tissue site in a patient.

In some examples herein, the electrode assemblies are configured to minimize impact to the ability of the expandable structure to transform between the delivery (e.g., the compressed or relatively low-profile) configuration and the deployed (e.g., expanded) configuration. In some examples, the electrode assemblies can be positioned, oriented, or otherwise configured with respect to portions of the expandable structure such that the expandable structure is able to transform between the delivery and the deployed configuration with minimal or no interference (e.g., mechanical interference) from electrode assemblies mechanically coupled to the expandable structure.

In some examples herein, the electrode assemblies can be formed using suitable processes or combination of processes (e.g., manufacturing processes) that are relatively more efficient or result in less manufacturing mistakes. In some examples herein, electrode assemblies are formed from a combination of processes (e.g., to form each of the electrically conductive portion and the electrically insulative portion of each respective electrode assembly). In some examples, the electrically conductive portion of the electrode assemblies is formed via a machining or stamping process. In some examples, the electrically insulative portion of the electrode assemblies described herein is formed via injection molding (e.g., insert molded or injection molding around and/or through a portion of the electrically conductive portion). Thus, the electrode assemblies described herein can include an electrically conductive portion formed from a first manufacturing processing (e.g., machining, stamping, and/or the like), and an electrically insulative portion formed from a second manufacturing processing (e.g., injection molding, extrusion, or the like).

Forming and/or assembling the electrode assemblies from at least a first manufacturing process and a second manufacturing process can facilitate use of relative simply geometries for at least the electrically conductive portion of the electrode assembly (e.g., since the more complex geometries use for the electrode attachment portion and/or electrically insulative portion of the electrode assemblies can be formed from the second manufacturing process). In some examples herein, the electrically conductive portions of the electrode assemblies can include relative simply geometries such as tubular (e.g., cylindrical), half-cylinder, ring-like, or the like. The relative simply geometries of the electrically conductive portions of the electrode assemblies can reduce cost and facilitate easier formation of the electrically conductive portion and assembly into the electrode assembly.

In some examples, an endovascular medical device system includes an elongated body configured to be introduced in a blood vessel of a patient; an expandable structure at a distal portion of the elongated body, the expandable structure includes an expandable body portion including a plurality of interconnected struts; and a plurality of electrode attachment elements, each electrode attachment element of the plurality of electrode attachment elements including one or more projections branching off of a strut of the plurality of interconnected struts; and one or more electrode assemblies mechanically coupled to the expandable structure via at least one electrode attachment element of the plurality of electrode attachment elements, each respective electrode assembly of the one or more electrode assemblies includes an electrically conductive portion; and an electrically insulative portion configured to receive the one or more projections, wherein each electrode attachment element of the plurality of electrode attachment elements is configured to orient a respective electrode assembly of the one or more electrode assemblies such that the electrically conductive portion faces radially outward from the expandable structure.

In some examples, a method of using a medical device system includes introducing a medical device into vasculature of a patient, the medical device includes an elongated body configured to be introduced in a blood vessel of a patient; an expandable structure at a distal portion of the elongated body, the expandable structure includes an expandable body portion including a plurality of interconnected struts; and a plurality of electrode attachment elements, each electrode attachment element of the plurality of electrode attachment elements including one or more projections branching off of a strut of the plurality of interconnected struts; and one or more electrode assemblies mechanically coupled to the expandable structure via at least one electrode attachment element of the plurality of electrode attachment elements, each respective electrode assembly of the one or more electrode assemblies includes an electrically conductive portion; and an electrically insulative portion configured to receive the one or more projections, wherein each electrode attachment element of the plurality of electrode attachment elements is configured to orient a respective electrode assembly of the one or more electrode assemblies such that the electrically conductive portion faces radially outward from the expandable structure; and advancing the medical device until the one or more electrode assemblies are at or near a target location in the vasculature of the patient.

In some examples, an electrode assembly includes an electrically conductive portion configured to transmit electrical signals, and an electrically insulative portion, the electrically insulative portion molded around a surface of the electrically conductive portion, wherein one or more of the electrically conductive portion and the electrically insulative portion defines a conductor hole configured to receive a conductor wire, and wherein the electrically insulative portion defines at least one fixation hole configured to receive a projection of an expandable structure to mechanically couple the electrode assembly to the expandable structure.

The examples described herein may be combined in any permutation or combination.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a conceptual diagram illustrating an example therapy system including an endovascular device configured to deliver electrical stimulation therapy to a target tissue site of a patient and/or sense a patient parameter from an endovascular location.

FIG. 2 is a functional block diagram illustrating components of an example medical device of the therapy system of FIG. 1.

FIG. 3 illustrates a distal portion of the example endovascular therapy system of FIG. 1 including an expandable structure and electrode assemblies carried by the expandable structure.

FIG. 4A and FIG. 4B illustrate an example electrode attachment element branching off of a strut of an example expandable structure.

FIG. 5 illustrates an example electrode attachment element including multiple projections branching away from the strut.

FIG. 6 illustrates an example electrode attachment element including multiple projections branching away from the strut.

FIG. 7A and FIG. 7B illustrate an example electrode assembly including an electrically conductive portion and an electrically insulative portion.

FIG. 8A and FIG. 8B illustrate an example electrode assembly including an electrically conductive portion and an electrically insulative portion.

FIG. 9A and FIG. 9B illustrate an example electrode assembly including an electrically conductive portion and an electrically insulative portion.

FIG. 10 is a flow diagram illustrating an example technique for introducing and advancing an endovascular device according to this disclosure.

Like reference characters denote like elements throughout the description and figures.

### DETAILED DESCRIPTION

This disclosure describes devices, systems, and methods relating to delivery of electrical stimulation therapy, such as vagus nerve stimulation (VNS), deep brain stimulation (DBS), and/or sensing one or more patient parameters (e.g., nerve activity from one more nerves, cardiac signals, muscle activation signals, brain signals and/or other physiological parameters, such as impedance, electroencephalogram (EEG), evoked potentials, local field potentials, etc.) from an endovascular location. Example endovascular locations that can be used for electrical stimulation therapy (e.g., VNS therapy) and/or sensing using the devices described herein include an internal jugular vein (IJV). Example endovascular locations that can be used to access the brain sites for electrical stimulation therapy (e.g., DBS) and/or sensing using the devices described herein include any suitable cranial blood vessel (also referred to herein as a cerebral blood vessel or neurovasculature, which can include a vein or an cranial artery), such as, but not limited to, the thalamostriate vein, the internal cerebral vein, the basal vein of Rosenthal, the inferior sagittal sinus, the superior sagittal sinus, or the anterior choroidal artery.

VNS has been proposed for use to manage one or more patient conditions, such as to control an inflammatory response in patients. Stimulating the vagus nerve may dampen the inflammatory response and associated cytokine response. In some examples, inflammatory cytokines are modulated up or down via stimulation. In addition, VNS may assist in stroke rehabilitation and limit ischemia reperfusion injury. After a myocardial infarct or stroke, reperfusion therapies (surgery or drugs) are given to restore blood flow. However, due to the restoration of blood, flow induced local damage occurs, including ischemia reperfusion injury. This injury may induce local accumulations of chemical mediators such as reactive oxygen species (ROS) production, inflammatory cytokines, bradykinin, etc., which can further affect inflammation. Such inflammatory compounds may trigger sensory signaling, which can lead to a reduced organ vagus activity and sympathetic overdrive. Vagus nerve stimulation may treat reperfusion damage as the inflammatory state may be lowered by increasing parasympathetic drive.

DBS has been proposed for use to manage one or more patient conditions. For example, DBS can be used to alleviate, and in some cases, eliminate symptoms associated with movement disorders, other neurodegenerative impairment, seizure disorders, psychiatric disorders (e.g., mood disorders), or the like. Movement disorders may be found in patients with Parkinson's disease, multiple sclerosis, and cerebral palsy, among other conditions, and can be associated with disease or trauma. DBS can be delivered to one or more target sites in a brain of a patient to help a patient with muscle control and minimize movement problems, such as rigidity, bradykinesia (i.e., slow physical movement), rhythmic hyperkinesia (e.g., tremor), nonrhythmic hyperkinesia (e.g., tics) or akinesia (i.e., a loss of physical movement).

In the case of seizure disorders, DBS can be delivered to one or more target sites in a brain of a patient to reduce the frequency or severity of seizures, or even help prevent the occurrence of seizures. In the case of psychiatric disorders, DBS can be delivered to help minimize or even eliminate symptoms associated with major depressive disorder (MDD), bipolar disorder, anxiety disorders, post-traumatic stress disorder, dysthymic disorder, or obsessive-compulsive disorder (OCD). DBS can also reduce the symptoms of Parkinson's disease, dystonia, or cerebellar outflow tremor.

While this disclosure is primarily directed to examples of VNS and/or sensing via applicable endovascular locations (e.g., the internal jugular vein), it should be understood that the devices, systems, and techniques may be adapted for DBS, other kinds of brain stimulation, peripheral nerve stimulation, or electrical stimulation and/or sensing of any nerve tissue that can be done via an endovascular location.

In the examples described herein, an endovascular therapy system includes one or more electrode assemblies that are carried by an expandable structure at a distal portion of an elongated body (e.g., a medical lead). The expandable structure is configured to transform between a delivery configuration (e.g., compressed configuration and/or low-profile configuration) and a deployed (e.g., expanded) configuration. The expandable structure (e.g., a stent, or stent-like structure) includes struts and a plurality of electrode attachment elements. The electrode attachment elements facilitate mechanical coupling of the one or more electrode assemblies to the expandable structure.

Although the term “electrode assembly” is used throughout this disclosure, such elements can also be referred to as an electrode or an electrode device.

Because the electrode assemblies described in this disclosure can be fabricated separately from the expandable structure, properties of the electrode assemblies can be selected and/or adjusted during fabrication, manufacturing, and/or assembly for a particular end use, and the electrode assemblies can subsequently be attached to the expandable structure via the electrode attachment elements. Each electrode attachment element, which may include and/or be affixed to a portion of one or more struts of the expandable structure, can include one or more structural features (e.g., projections) configured to facilitate mechanical coupling of electrode assemblies to the expandable structure. Each electrode assembly is configured to receive the structural features (e.g., projections) of respective one or more electrode attachment elements to mechanically couple the respective electrode assembly to the expandable structure. The electrode attachment elements described herein can be configured to minimize or even prevent rotation of the electrode assemblies with respect to the struts of the expandable structure.

Minimizing, or even preventing, rotation of electrode assemblies with respect to struts can facilitate directional orientation of electrode assemblies with respect to the expandable structure. For example, by minimizing, or even preventing, rotation of electrode assemblies with respective to struts, an electrically conductive portion of each electrode assembly can be configured to face radially outward from the expandable structure (e.g., from a central longitudinal axis of the expandable structure) with little or no possibility of the electrically conductive face being oriented radially inward, such as away from a blood vessel wall. Said another way, the system can bias transmissions of electrical signals to and/or sensing from tissue surrounding blood vessel as compared to radially inward from the blood vessel wall. In this way, the system is configured to facilitate directional electrical stimulation therapy and/or directional sensing, such as in a direction radially outward from the expandable structure and/or a blood vessel and towards a blood vessel wall and/or in a specific direction away from the expandable structure. The directional electrical stimulation facilitated by the electrode assemblies described herein can help direct electrical stimulation signals to a specific target tissue site to enhance therapy efficacy and reduce possible adverse side effects from stimulating unintended tissue sites (e.g., particular nerves or brain targets). Further, by fixing the particular surface of the electrode that faces radially outward towards a vessel wall, the systems described in this disclosure may be relatively more efficient (e.g., in terms of power used and/or power lost) during electrical stimulation therapy and/or sensing, such as compared to other types of systems that do not fix or orient conductive surfaces in a particular direction.

In some examples herein, electrode assemblies include structural features to facilitate mechanical coupling of the electrode assemblies to expandable structures (e.g., stents or stent-like structures). In some examples, the structural features enable the electrode assemblies to receive portions (e.g., mating features, including projections) of the electrode attachment elements of the expandable structure to facilitate mechanical coupling of the electrode assemblies to the expandable structure. Thus, the features of the electrode assemblies can be considered complementary or mating structural features to the structural features of the electrode attachment elements.

In some examples herein, electrode assemblies include structural features for coupling to conductor wires (e.g., to facilitate electrical coupling of the electrode assemblies to a medical device). In some examples, electrode assemblies are configured to receive and/or electrically couple to one or more conductor wires (e.g., conductor wires that are configured to electrically couple the electrode assemblies to a medical device). In some examples, electrode assemblies include pass-through holes, such as for routing conductor wires to different electrode assemblies.

In some examples, the structural features that facilitate mechanical coupling of the electrode assemblies to the expandable structures are also configured to electrically insulate a given electrode assembly from the expandable structure. In other words, structural features of the electrode assemblies described herein can facilitate mechanical coupling (e.g., physical connection) of the electrode assemblies to the expandable structure as well as facilitate the secondary purpose of electrically insulating the electrode assemblies from the expandable structure. This dual purpose of facilitating mechanical coupling and facilitating electrical insulation can reduce or even eliminate extra material and/or manufacturing steps that would otherwise need to be incorporated into the electrode assemblies to facilitate the mechanical coupling and electrical insulation functions. In addition, reducing or even eliminating other electrically insulative material can help maintain a relatively low profile medical device suitable for endovascular delivery to a target tissue site in a patient.

In some examples, electrode assemblies include electrically insulative portions that are configured to mechanically couple to the expandable structure (e.g., via electrode attachment elements), such that when the electrode assemblies are mechanically coupled to the expandable structure, an electrically conductive portion of a respective electrode assembly is electrically insulated from the expandable structure. By electrically insulating the electrically conductive portion of each electrode assembly from the expandable element, loss of electrical power due to dissipation to the expandable structure (which may be electrically conductive) is minimized. Further, by electrically insulating the electrically conductive portion of each electrode assembly from the expandable element, the directionality of electrode assemblies (e.g., the radial direction outward faced by one or more electrode assemblies) can be maintained. In addition, in some examples, a plurality of electrode assemblies are mechanically coupled to the same expandable structure, and electrically insulating the electrically conductive portion of each electrode assembly from the expandable structure can help prevent inadvertent shorting between the electrode assemblies and, for example, maintain the ability to independent control (e.g., activate) each electrode assembly for sensing and/or electrical stimulation therapy delivery.

In some examples herein, the electrode assemblies are configured to minimize impact to the ability of the expandable structure to transform between the delivery (e.g., the compressed or relatively low-profile) configuration and the deployed (e.g., expanded) configuration. In some examples, the electrode assemblies can be positioned, oriented, or otherwise configured with respect to portions of the expandable structure such that the expandable structure is able to transform between the delivery and the deployed configuration without interference (e.g., mechanical interference) from electrode assemblies mechanically coupled to the expandable structure.

In some examples herein, the electrode assemblies can be formed using suitable processes or combination of processes (e.g., manufacturing processes) that are relatively more efficient or result in less manufacturing mistakes. In some examples herein, electrode assemblies are formed from a combination of processes (e.g., to form each of the electrically conductive portion and the electrically insulative portion of each respective electrode assembly). In some examples, the electrically conductive portion of the electrode assemblies is formed via a machining or stamping process. In some examples, the electrically insulative portion of the electrode assemblies described herein is formed via injection molding (e.g., insert molded or injection molding around and/or through a portion of the electrically conductive portion). Thus, the electrode assemblies described herein can include an electrically conductive portion formed from a first manufacturing processing (e.g., machining, stamping, and/or the like), and an electrically insulative portion formed from a second manufacturing processing (e.g., injection molding, extrusion, or the like).

Forming and/or assembling the electrode assemblies from at least a first manufacturing process and a second manufacturing process can facilitate use of relative simply geometries for at least the electrically conductive portion of the electrode assembly (e.g., since the more complex geometries use for the electrode attachment portion and/or electrically insulative portion of the electrode assemblies can be formed from the second manufacturing process). In some examples herein, the electrically conductive portions of the electrode assemblies can include relative simply geometries such as tubular (e.g., cylindrical), half-cylinder, ring-like, or the like. The relative simply geometries of the electrically conductive portions of the electrode assemblies can reduce cost and facilitate easier formation of the electrically conductive portion and assembly into the electrode assembly.

In some examples, a medical device is configured to deliver electrical stimulation and/or sense a patient parameter via the electrode assemblies of the endovascular device. The electrode assemblies may be carried by or otherwise disposed on an expandable structure, which may be configured to orient the electrode assemblies and/or anchor the electrode assemblies at a particular location in the vasculature of the patient.

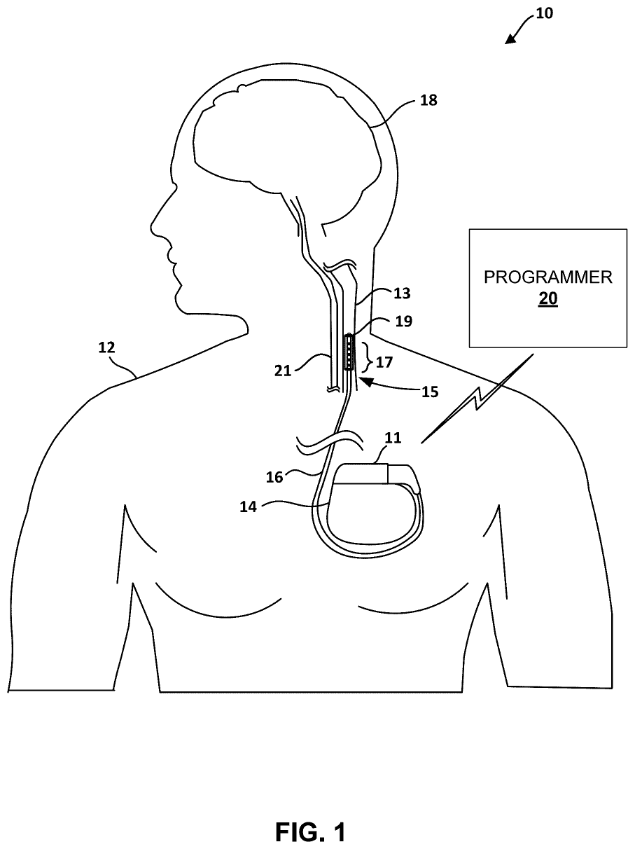

FIG. 1 is a conceptual diagram illustrating an example therapy system 10 configured to deliver electrical stimulation therapy to a target tissue site of a patient 12 or sense a patient parameter from an endovascular location. Patient 12 ordinarily will be a human patient. In some cases, however, therapy system 10 is applied to other mammalian or non-mammalian non-human patients. Therapy system 10 includes a medical device 14 and an endovascular device 16. In the example shown in FIG. 1, medical device 14 is configured to deliver electrical stimulation therapy (e.g., VNS) to a vagus nerve 21 of patient 12 and/or sense bioelectrical signals via one or more electrode assemblies 17. However, in other examples, therapy system 10 and/or medical device 14 is configured to deliver electrical stimulation therapy (e.g., DBS) to brain 18 of patient 12 and/or sense bioelectrical brain signals in brain 18 via electrode assemblies 17.

In the example shown in FIG. 1, endovascular device 16 is positioned in a jugular vein 13 of patient 12 such that one or more electrode assemblies 17 are located proximate to a target tissue site. In particular, electrode assemblies 17 are positioned to deliver electrical stimulation therapy to and/or sense signals from nerves surrounding jugular vein 13, including (but not limited to) vagus nerve 21. Endovascular device 16 includes an expandable structure 19 at a distal portion 15 of endovascular device 16 which may help hold electrode assemblies 17 in apposition with a vessel wall (e.g., of jugular vein 13). In some examples, expandable structure 19 is at a distalmost end of endovascular device 16. In other examples, expandable structure is proximal to a distal end of endovascular device 16. Medical device 14 can provide electrical stimulation to one or more regions surrounding jugular vein 13 in order to manage a condition of patient 12, such as to mitigate the severity or duration of the patient condition.

Endovascular device 16 includes any suitable medical device configured to deliver electrical stimulation signals to tissue proximate electrode assemblies 17. For example, endovascular device 16 can be a medical lead, a catheter, a guidewire, or another elongated body carrying electrode assemblies 17 and configured to be electrically coupled to medical device 14 via an electrically conductive pathway (e.g., via one or more conductor wires) that runs between medical device 14 and electrode assemblies 17. Endovascular device 16 has any suitable length that enables connection to medical device 14 either directly or indirectly, e.g., a length of 150 centimeters (cm) to 250 cm, such as 200 cm. Further, endovascular device 16 has a suitable length (e.g., as measured along a longitudinal axis of endovascular device 16) for accessing a target tissue site within the patient from a vascular access point. In examples in which endovascular device 16 accesses the jugular vein 13 and/or vasculature in a brain 18 of patient 12 from a femoral artery access point at the groin of the patient, endovascular device 16 has a length of about 100 cm to about 200 cm, although other lengths may be used. However, other access points may be used to introduce endovascular device 16 into vasculature of a patient, such as, but not limited to, a radial artery.

As used herein, “about” may indicate the exact value or nearly the exact value to the extent permitted by manufacturing tolerances. “About” can also refer to a certain percentage of the recited value (e.g., within about 1%, 5%, or 10%).

Endovascular device 16 is configured to be introduced in the vasculature of patient 12, such as to access jugular vein 13 and/or relatively more distal locations in a patient, such as the middle cerebral artery (MCA) in a brain of a patient. Endovascular device 16 may include an elongated body that is structurally configured to be relatively flexible, pushable, and relatively kink- and buckle-resistant, so that it may resist buckling when a pushing force is applied to a relatively proximal portion to advance endovascular device 16 distally through vasculature, and so that it may resist kinking when traversing around a tight turn in the vasculature. Kinking and/or buckling of may hinder a clinician's efforts to push the elongated body distally, e.g., past a turn. In some examples, endovascular device 16 includes one or more radiopaque components (e.g., platinum bands) proximate electrode assemblies 17 and/or expandable structure 19.

Instead of or in addition to the elongated body of endovascular device 16 being configured for intravascular navigation to a cerebral blood vessel to deliver electrical stimulation therapy or sense a patient parameter, endovascular device 16 can be navigated through vasculature (e.g., to jugular vein 13, brain 18, or other target tissue sites) with the aid of a guide member. The guide member can include an outer catheter, an inner catheter, a guide extension catheter, a guidewire, or the like or combination thereof.

In some examples, more than one endovascular device 16 is implanted within patient 12 to provide stimulation to and/or sense multiple anatomical regions, including one or more of both the left and right jugular veins, as well as in locations of brain 18. For example, two or more of endovascular device 16, which may be paired with one or more of medical device 14, may be configured of bilateral stimulation and/or sensing (e.g., of the left jugular vein and a right jugular vein). Endovascular device 16, including electrode assemblies 17 and/or expandable structure 19, can be implanted in a blood vessel for chronic therapy delivery and/or chronic sensing (e.g., on the order of months or even years) or for more temporary therapy delivery and/or sensing (e.g., on the order of days, such as less than a month or less than 6 months). Temporary therapy delivery may include one or more acute periods (e.g., trial periods), such as to determine, evaluate, or confirm an efficacy of stimulation and/or sensing, and/or to select electrical stimulation parameters for chronic therapy delivery.

The electrical stimulation therapy described herein (e.g., VNS, DBS, or the like) may be used to treat various patient conditions, such as, a variety of illnesses including, but not limited to: reperfusion damage, cardiac ischemia, brain ischemia, stroke, traumatic brain injury, surgical or non-surgical acute kidney injury, inability of the intestine (bowel) to contract normally and move waste out of the body, postoperative ileus, postoperative cognitive decline or postoperative delirium, asthma, sepsis, bleeding control, myocardial infarction reduction, dysmotility, and obesity. Treating any of these diseases may improve patient outcomes by shortening length of hospital stays and reducing medical costs.

The vasculature into which endovascular device 16 may be inserted and/or guided includes, but is not limited to, veins or arteries. For example, endovascular device 16 can be navigated from a vasculature access site (e.g., in the femoral artery, the radial artery, or another suitable access site) to one or more of a jugular vein (e.g., internal jugular vein and/or external jugular vein), a carotid artery (e.g., internal carotid artery, external carotid artery, and/or common carotid artery), as well as brain targets including the thalamostriate vein, the internal cerebral vein, the basal vein of Rosenthal, the inferior/superior sagittal sinus, the anterior choroidal artery, or any related combinations thereof.

A clinician can also select a particular blood vessel to position electrode assemblies 17 within, such as to avoid certain regions to minimize or even eliminate adverse effects. For example, electrode assemblies 17 can be oriented or positioned relative to vagus nerve 21 to avoid inadvertently providing electrical stimulation to anatomical regions (e.g., undesired anatomical regions) near the targeted anatomical region.

In some examples, endovascular device 16 is configured to be delivered to one or more target sites in vasculature of patient 12. Thus, rather than introducing endovascular device 16 into tissue in close proximity with vagus nerve 21 through an incision in the neck or chest area of patient 12, endovascular device 16 is configured to be navigated proximate to a target electrical stimulation site via vasculature of patient 12. The endovascular delivery of endovascular device 16 to target sites can help minimize the invasiveness of therapy system 10.

In some examples, one or more electrode assemblies 17 are positioned on (e.g., coupled to, defined by, or otherwise carried by) expandable structure 19 of endovascular device 16, which is configured to expand radially outwards from a relatively low-profile (e.g., radially compressed) delivery configuration to a deployed configuration. This may enable electrode assemblies 17 to be held in apposition with a blood vessel wall, promote tissue ingrowth around electrode assemblies 17 along the vessel wall (while still leaving a patent lumen to enable blood flow through the blood vessel, through expandable structure 19, despite implantation of endovascular device 16), which can reduce the overall power needed to deliver efficacious electrical stimulation therapy to a target tissue site, and help secure electrode assemblies 17 in place in the blood vessel for chronic therapy delivery.

Medical device 14 can be an external medical device or an implantable medical device that includes electrical stimulation circuitry configured to generate and deliver electrical stimulation therapy to patient 12 and/or sensing circuitry configured to sense a patient parameter (e.g., a physiological signal) via one or more electrode assemblies 17 of endovascular device 16. In the example shown in FIG. 1, endovascular device 16 is directly or indirectly mechanically and electrically coupled to medical device 14 via a header 11 of medical device 14, which defines a plurality of electrical contacts in one or more feedthrough portions for electrically coupling electrode assemblies 17 to electrical stimulation generation circuitry and/or sensing circuitry within medical device 14. In some examples, header 11 includes multiple feedthrough portions, which may be respectively configured for receiving one of multiple portions of endovascular device 16. Header 11 may also be referred to as a connector block or connector of medical device 14. Endovascular device 16 may be coupled to header 11 with the aid of a lead extension. However, in some examples, a lead extension is not used between header 11 and endovascular device 16, and endovascular device is directly mechanically and/or electrically connected to medical device 14 via header 11.

In some examples, medical device 14 is configured to be implanted in patient 12 in any suitable location, such as a location in a pectoral region. In other examples, medical device 14 is configured to be external to patient 12. Endovascular device 16 may be, for example, implanted within a vein (e.g., jugular vein 13) and one or more proximal wires/leads can remain within the venous system until they exit the venous system, such as through the subclavian vein in the chest or the internal jugular vein in the neck for implant in the pectoral region. In yet other examples, some or all of medical device 14 is configured to be implanted in the vasculature, e.g., as part of endovascular device 16.

As shown in FIG. 1, system 10 may also include a programmer 20, which may be a handheld device, portable computer, or workstation that provides a user interface to a user, for example a clinician or other user, such as a patient. The user may interact with the user interface to program electrical stimulation parameters for medical device 14.

With the aid of programmer 20 or another computing device, a clinician may select values for therapy parameters for controlling therapy delivery by therapy system 10. The values for the therapy parameters may be organized into a group of parameter values referred to as a “therapy program” or “therapy parameter set.” “Therapy program” and “therapy parameter set” are used interchangeably herein. In the case of electrical stimulation, the therapy parameters may include a combination of activated electrode assemblies (also referred to herein as an electrode combination), a power, and an amplitude, which may be a current or voltage amplitude, and, if medical device 14 delivers electrical pulses, a pulse width, and a pulse rate for stimulation signals to be delivered to the patient. Other example therapy parameters include a slew rate, duty cycle, and phase of the electrical stimulation signal.

A combination of active electrode assemblies may include a selected subset of one or more electrode assemblies 17 located on one or more of implantable endovascular device 16 coupled to medical device 14. The electrode combination may also refer to the polarities of the electrode assemblies in the selected subset. By selecting particular combination of active electrode assemblies, a user may target particular tissue sites (e.g., anatomic structures) within patient 12. In addition, by selecting values for slew rate, duty cycle, phase amplitude, pulse width, and/or pulse rate, the user can attempt to generate an efficacious therapy for patient 12 that is delivered via the selected subset of active electrode assemblies.

Whether programmer 20 is configured for clinician or patient use, programmer 20 may be configured to communicate with medical device 14 or any other computing device via wireless or a wired communication. Programmer 20, for example, may communicate via wireless communication with medical device 14 using radio frequency (RF) telemetry techniques. Programmer 20 may also communicate with another programmer or computing device via a wired or wireless connection using any of a variety of local wireless communication techniques, such as RF communication according to the 802.11 or Bluetooth specification sets, infrared communication according to the Infrared Data Association (IRDA) specification set, or other standard or proprietary telemetry protocols. Programmer 20 may also communicate with another programming or computing device via a wired or wireless communication technique.

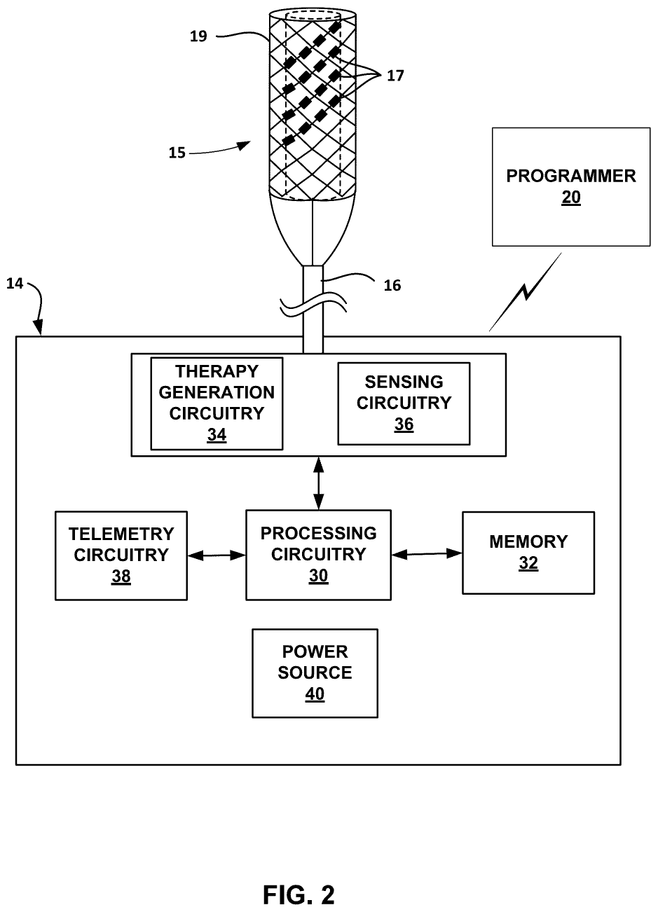

In some examples, in addition to or instead of delivering electrical stimulation to a target location (e.g., vagus nerve 21), medical device 14 or another device is configured to sense one or more patient parameters, such as bioelectrical signals, either using electrode assemblies 17 or other types of sensors that are carried by endovascular device 16. Bioelectric (also referred to herein as bioelectrical) signals can be sensed, and indications of sensed signals can be used by clinicians to make clinically relevant decision. In other examples, sense bioelectric signals are used as part of continuous feedback system in which medical device 14 adjusts one or more therapy parameter values based on sensed bioelectrical signals. Example bioelectric signals are described in further detail below with reference to FIG. 2.

In some examples, medical device 14 is configured to generate and deliver a suitable electrical stimulation signal, which can be a continuous time signal (e.g., a sinusoidal waveform or the like) or a plurality of pulses. In some examples, the electrical stimulation waveform generated by medical device 14 and delivered by one or more of electrode assemblies 17 is a charge balanced, biphasic waveform. In some examples, such an electrical stimulation waveform consists of periodic pulses or otherwise include periodic pulses, or can include a continuous time waveform.

As noted above, in some examples, one or more electrode assemblies 17 are positioned on expandable structure 19. In some examples, one or more sensors that are different from electrode assemblies 17 are positioned on the same expandable structure (e.g., expandable structure 19) as one or more electrode assemblies 17 or on a different expandable structure (e.g., a structure similar to or different from expandable structure 19) of endovascular device 16. Expandable structure 19 can have any suitable configuration that enables endovascular device 16 to assume a relatively low-profile configuration (also referred to herein as a “delivery” or “compressed” configuration in some examples) to facilitate delivery through vasculature to a target tissue site and expand radially outwards (relative to a central longitudinal axis of endovascular device 16) to position the one or more electrode assemblies 17 closer to target tissue.

In some examples, expandable structure 19 is configured to expand radially outwards with sufficient force and to a cross-sectional dimension (e.g., a diameter) sufficient to position the one or more electrode assemblies 17 in apposition with a blood vessel wall. Positioning one or more electrode assemblies 17 in apposition with a blood vessel wall may help promote tissue ingrowth around electrode assemblies 17, which can reduce the impedance and the overall power needed to deliver efficacious electrical stimulation therapy to a target tissue site, and help secure electrode assemblies 17 in place in the blood vessel for chronic (e.g., on the order of months or even years) therapy delivery. Fixing endovascular device 16 in place within the blood vessel via the tissue ingrowth or, in some examples, using another fixation structures/anchoring mechanisms, such as tines, coils barbs, or the like, can also help reduce the possibility of thrombosis.

Expandable structure 19 can be configured to expand radially outwards using any suitable technique and configuration. In some examples, expandable structure 19 includes a shape memory (e.g., nitinol) material that enables expandable structure 19 to assume a predetermined shape in the absence of a force (e.g., a compressive or tensile force) holding expandable structure 19 in a relatively low-profile delivery configuration. For example, expandable structure 19 can be configured to expand radially outwards upon deployment from an outer sheath (e.g., an outer catheter), or upon the proximal withdrawal of a straightening element (e.g., a guidewire or a mandrel) positioned in an inner lumen of the endovascular device 16. In some examples, expandable structure 19 is configured to expand radially outwards in response to proximal movement of a pull member attached to a distal portion of the endovascular device 16, in response to a distal movement of an elongated control member attached to the expandable structure, or with the aid of a balloon or the like.

Expandable structure 19 can have any suitable configuration in its deployed (e.g., expanded) configuration. In some examples herein, expandable structure 19 includes a plurality of struts (e.g., interconnected struts) to form a structure (e.g., an expandable body portion) configured to expand radially outward (e.g., from a central longitudinal axis of expandable structure 19). For example, expandable structure 19 can include a tubular member and/or a basket, include one or more splines or arms configured to expand radially outwards, define one or more loops, define a helical or spiral element, or the like or combinations thereof, when in the deployed configuration. One or more expandable structures 19 may be disposed at various positions along endovascular device 16 (e.g., at one or more longitudinal positions along endovascular device 16). Expandable structure 19 can be formed from a plurality of structural elements (e.g., braided or coupled together) or can be a unitary structure (e.g., a laser cut nitinol tube). In some examples, expandable structure 19 is referred to herein as having a stent-like structure.

In addition to, or instead of, chronic therapy delivery and/or chronic sensing, example devices, systems, and methods described herein can be used for more temporary applications. In some examples, a first endovascular device (e.g., configured like endovascular device 16 or having another configuration) is configured to be operated in an acute (e.g., temporary) mode for an acute period, which may be a trial mode for a trial period, to determine, evaluate, or confirm an efficacy of stimulation and/or sensing. For example, endovascular device 16 (as well as electrode assemblies 17, medical device 14, processing circuitry, etc.) may be configured to operate in the trial mode to determine the efficacy of one or more stimulation parameter values and/or one or more sensing parameters. After the acute period (e.g., trial period), the first endovascular device may be removed, and a second endovascular device (e.g., configured like endovascular device 16 or having another configuration) configured to operate in a chronic mode may be implanted for a chronic period for chronic (e.g., long term, or permanent) stimulation therapy or sensing. In some examples, a first endovascular device (e.g., for use in the acute mode and/or trial mode) is configured to be implanted and subsequently removed after the acute period and/or trial period.

An acute period and/or trial period has a shorter intended duration than a chronic period, though the ultimate length of the chronic period may be less than an intended duration due to one or more factors, such as a patient response that requires shortening the chronic period relative to the intended duration of the chronic period. In some examples, the acute period (e.g., trial period) includes a period length on the order of minutes (e.g., 1 minute, 2 minutes, 3 minutes, 5 minutes, 30 minutes, 45 minutes, etc.), on the order of hours (e.g., 1 hour, 2 hours, 5 hours, 12 hours, etc.), on the order of days (e.g., 1 day, 2 days, 3 days, etc.), on the order of weeks (e.g., 1 week, 2 weeks, 3 weeks, etc.) on the order of months (e.g., 1 month, 2 months, 3 months, etc.), or longer. In some examples, one or more endovascular devices may be used for multiple acute periods and/or trial periods (e.g., successive acute period and/or trial periods) for determining an efficacy of one or more stimulation parameters and/or one or more sensing parameters.

Endovascular device 16 may have any suitable configuration for delivering electrical stimulation to a target tissue site in patient 12 or sensing a patient parameter from an endovascular location (e.g., jugular vein 13). In some examples, endovascular device includes a first subset of electrode assemblies of electrode assemblies 17 configured for delivering electrical stimulation therapy and a second subset of electrode assemblies of electrode assemblies 17 configured to for sensing one or more patient parameters. In some examples, some or all of electrode assemblies 17 are configured for both electrical stimulation therapy and for sensing one or more patient parameters. Endovascular device 16 can include any suitable number of electrode assemblies 17 and/or combination of different kinds of electrode assemblies.

In some examples, electrode assemblies 17 include electrode assemblies that have different form factors and/or structural features. For example, electrode assemblies 17 can include a first electrode type (e.g., an electrode assembly that includes a conductor pass-through hole to facilitate connection of a conductor wire to a second, different electrode assembly), a second electrode type (e.g., an electrode assembly that does not include a conductor pass-through hole), or any suitable combination thereof.

FIG. 2 is a functional block diagram illustrating components of an example medical device 14, which is configured to generate and deliver electrical stimulation therapy to patient 12 and, in some examples, sense one or more patient parameters, such as bioelectrical signals or other physiological parameter of patient 12. Medical device 14 includes processing circuitry 30, memory 32, therapy generation circuitry 34, sensing circuitry 36, telemetry circuitry 38, and power source 40.

Therapy generation circuitry 34 includes any suitable configuration (e.g., hardware) configured to generate and deliver electrical stimulation signals to target tissue (e.g., vagus nerve 21) in patient 12. Processing circuitry 30 is configured to control therapy generation circuitry 34 to generate and deliver electrical stimulation therapy via electrode assemblies 17 of endovascular device 16. The therapy parameter values may be selected based on the patient condition being addressed, as well as the target tissue site in patient 12 for the electrical stimulation therapy. The electrical stimulation therapy can be provided via stimulation signals of any suitable form, such of stimulation pulses or continuous-time signals (e.g., sine waves).

Sensing circuitry 36 is configured to sense a physiological parameter of a patient. Sensing circuitry 36 may include any sensing hardware configured to sense a physiological parameter of a patient, such as, but not limited to, one or more electrode assemblies, optical receivers, pressure sensors, or the like. The one or more sensing electrode assemblies can be the same or different from electrode assemblies 17 configured to deliver electrical stimulation therapy. In some examples, processing circuitry 30 stores the sensed physiological parameters in memory 32 or transmits the sensed parameters to another device via telemetry circuitry 38. In addition, in some examples, processing circuitry 30 can use the sensed physiological signals to control therapy delivery by therapy generation circuitry 34, e.g., the timing of the therapy delivery or one or more characteristics (e.g., parameters values) of the electrical simulation signal generated by therapy generation circuitry 34.

In some examples, sensing circuitry 36 is configured to sense a bioelectrical signal, which otherwise may be referred to as a patient parameter, via one or more electrode assemblies 17 (e.g., all or a subset of electrode assemblies 17). Thus, electrode assemblies 17 can be configured to receive or transmit energy (e.g., current). In some examples, such as those in which electrode assemblies 17 are placed proximate vagus nerve 21 (FIG. 1), example bioelectrical signals include muscle activation signals (e.g., laryngeal muscle activation), electrocardiogram (ECG), intracardiac electrogram (EGM), electromyogram (EMG). In other examples, such as those in which electrode assemblies 17 are placed in or otherwise proximate brain 18, example bioelectrical signals include brain signals such as an EEG signal, an electrocorticogram (ECoG) signal, a signal generated from measured field potentials within one or more regions of brain 18, action potentials from single cells within brain 18 (referred to as “spikes”), or evoked potentials. Determining action potentials of single cells within brain 18 may require resolution of bioelectrical signals to the cellular level and provides fidelity for fine movements, i.e., a bioelectrical signal indicative of fine movements (e.g., slight movement of a finger).

In examples in which endovascular device 16 is configured to sense an evoked potential, endovascular device 16 may also be configured to generate a stimulus (e.g., via therapy generation circuitry 34, alone or in combination with processing circuitry 30) to elicit the evoked potential. For example, endovascular device 16 can generate and deliver electrical stimulation to tissue in brain 18 and sense an evoked compound action potential (ECAP). An ECAP is synchronous firing of a population of neurons which occurs in response to the application of a stimulus including, in some cases, an electrical stimulus by endovascular device 16. The ECAP may be detectable as being a separate event from the stimulus itself, and the ECAP may reveal characteristics of the effect of the stimulus on the tissue.

In some examples, sensing circuitry 36 and/or processing circuitry 30 includes signal processing circuitry configured to perform any suitable analog conditioning of the sensed physiological signals. For example, sensing circuitry 36 may communicate to processing circuitry 30 an unaltered (e.g., raw) signal. Processing circuitry 30 may be configured to modify a raw signal to a usable signal by, for example, filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof. In some examples, the conditioned analog signals are processed by an analog-to-digital converter of processing circuitry 30 or other component to convert the conditioned analog signals into digital signals. In some examples, processing circuitry 30 operates on the analog or digital form of the signals to separate out different components of the signals. In some examples, sensing circuitry 36 and/or processing circuitry 30 performs any suitable digital conditioning of the converted digital signals, such as low pass, high pass, band pass, notch, averaging, or any other suitable filtering, amplifying, performing an operation on the signal, performing any other suitable digital conditioning, or any combination thereof. Additionally or alternatively, sensing circuitry 36 may include signal processing circuitry to modify one or more raw signals and communicate to processing circuitry 30 one or more modified signals.

In some examples, processing circuitry 30, alone or in combination with therapy generation circuitry 34 and/or sensing circuitry 36, is configured to operate medical device 14 (including electrode assemblies 17, endovascular device 16, etc.) for more acute (e.g., temporary) applications, such as in a trial mode for a trial period to determine an efficacy of electrical stimulation or sensing. As described above, a trial mode can include a trial period of stimulation and/or sensing to determine, evaluate, or confirm an efficacy of stimulation and/or sensing. In some examples, processing circuitry 30, alone or in combination with therapy generation circuitry 34 and/or sensing circuitry 36, is configured to deliver electrical stimulation therapy and/or sense a patient parameter during the trial period. In some examples, processing circuitry 30 is configured to determine, evaluate, or confirm an efficacy of stimulation and/or sensing. For example, processing circuitry 30 may determine one or more therapy parameters for chronic stimulation and/or sensing based on the trial period.

Although shown as part of medical device 14 in FIG. 2, in other examples, sensing circuitry 36 is part of a device separate from medical device 14. For example, sensing circuitry 36 can be part of an implantable sensing device implanted in patient 12.

Processing circuitry 30, as well as other processors, processing circuitry, controllers, control circuitry, and the like, described herein, may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, processing circuitry 30 includes multiple components, such as any combination of one or more microprocessors, one or more DSPs, one or more ASICs, or one or more FPGAs, as well as other discrete or integrated logic circuitry, and/or analog circuitry.

Memory 32 is configured to store program instructions, such as software, which may include one or more program modules, which are executable by processing circuitry 30. When executed by processing circuitry 30, such program instructions may cause processing circuitry 30 to provide the functionality ascribed to processing circuitry 30 herein. The program instructions may be embodied in software and/or firmware. Memory 32 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

Processing circuitry 30 is configured to control telemetry circuitry 38 to send and receive information. Telemetry circuitry 38, as well as telemetry modules in other devices described herein, such as programmer 20 (FIG. 1), may accomplish communication by any suitable communication techniques, such as RF communication techniques. In addition, telemetry circuitry 38 may communicate with external medical device programmer 20 via proximal inductive interaction of medical device 14 with programmer 20. Accordingly, telemetry circuitry 38 may send information to external programmer 20 on a continuous basis, at periodic intervals, or upon request from medical device 14 or programmer 20.

Power source 40 is configured to deliver operating power to various components of medical device 14. Power source 40 may include a small rechargeable or non-rechargeable battery and a power generation circuit to produce the operating power. Recharging may be accomplished through proximal inductive interaction between an external charger and an inductive charging coil within medical device 14. In some examples, power requirements may be small enough to allow medical device 14 to utilize patient motion and implement a kinetic energy-scavenging device to trickle charge a rechargeable battery. In other examples, traditional batteries may be used for a limited period of time.

In some examples, endovascular device 16 is configured to be a standalone electrical stimulation device and can include one or more elements of medical device 14 shown in FIG. 2.

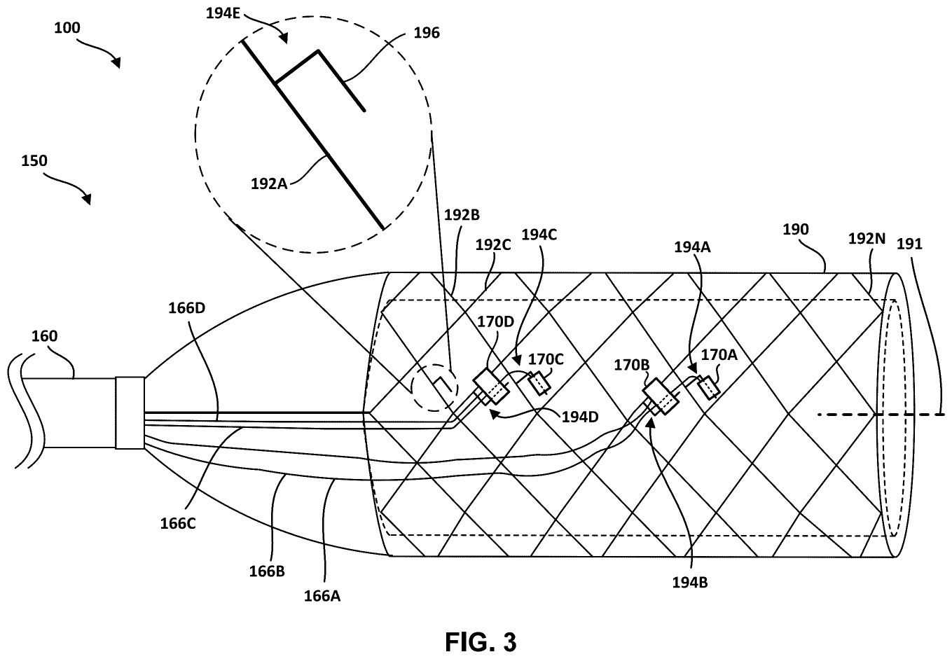

FIG. 3 illustrates an example endovascular therapy system 100, which is an example of therapy system 10 of FIG. 1. Endovascular therapy system 100 includes a medical lead 160 and an expandable structure 190 at a distal portion 150 of medical lead 160. As shown, endovascular therapy system 100 includes electrode assembly 170A, electrode assembly 170B, electrode assembly 170C, and electrode assembly 170D, collectively referred to herein as electrode assemblies 170. Medical lead 160, expandable structure 190, distal portion 150, and electrode assemblies 170 are examples of endovascular device 16, expandable structure 19, distal portion 15, and electrode assemblies 17 of FIG. 1, respectively.

Medical lead 160 can have any suitable configuration, and may be configured according to the description of endovascular device 16 of FIG. 1. In some examples, medical lead 160 includes a lead body that is configured to be at least partially positioned (e.g., implanted) in vasculature of patient 12. In some examples, lead 160 includes an electrically insulative material covering at least some portions of lead 160. The insulative material can be configured to electrically insulate portions of conductor wires 166 that run along the length of lead 160. Expandable structure 190 can be carried by or otherwise affixed to medical lead 160 using any suitable technique. In some examples, expandable structure 190 is welded to a portion of medical lead 160. In some examples a band (e.g., a marker band) of material or ring is configured to mechanically couple expandable structure 190 to lead 160. In general, expandable structure 190 can be positioned at a distal portion 150 of medical lead 160. In some examples, expandable structure 190 is positioned at a distal-most end of lead 160.

In some examples, expandable structure 190 is configured to position and/or orient electrode assemblies 170 within vasculature of a patient. In some examples, electrode assemblies 170 are carried by and/or disposed on expandable structure 190, and expandable structure 190 is configured to transform from a relatively low-profile delivery configuration to a deployed configuration in a blood vessel of a patient (e.g., within jugular vein 13 of patient 12 as discussed in relation to FIG. 1, and/or within a cranial blood vessel of patient 12). Expandable structure 190 is an example of expandable structure 19 of FIG. 1, and can include any suitable shape and materials. In some examples, expandable structure 190 includes one or more of a self-expanding stent, a self-expanding coil, or another suitable expandable structure that includes one or more struts as described herein.

Expandable structure 190 can have any suitable configuration for positioning electrode assemblies 170 for delivering stimulation therapy and/or sensing one or more patient parameters of patient 12 from an endovascular location. In some examples, as shown in FIG. 3, expandable structure 190 includes an expandable body portion that includes a plurality of interconnected struts 192 (shown individually as strut 192A, strut 192B, strut 192C . . . strut 192N, but collectively referred to herein as struts 192). In some examples, struts 192 are interconnected to form a tubular (e.g., stent-like) structure. Expandable structure 190 defines a central longitudinal axis 191. In some examples, expandable structure 190 is configured to expand (e.g., self-expand and/or via an expansion mechanism such as a balloon) radially outward from central longitudinal axis 191 to a deployed configuration, such as to position electrode assemblies 170 into apposition with a blood vessel wall (e.g., for delivering electrical stimulation therapy to tissue of patient 12 proximate the blood vessel and/or sensing a patient parameter from a location within the blood vessel).

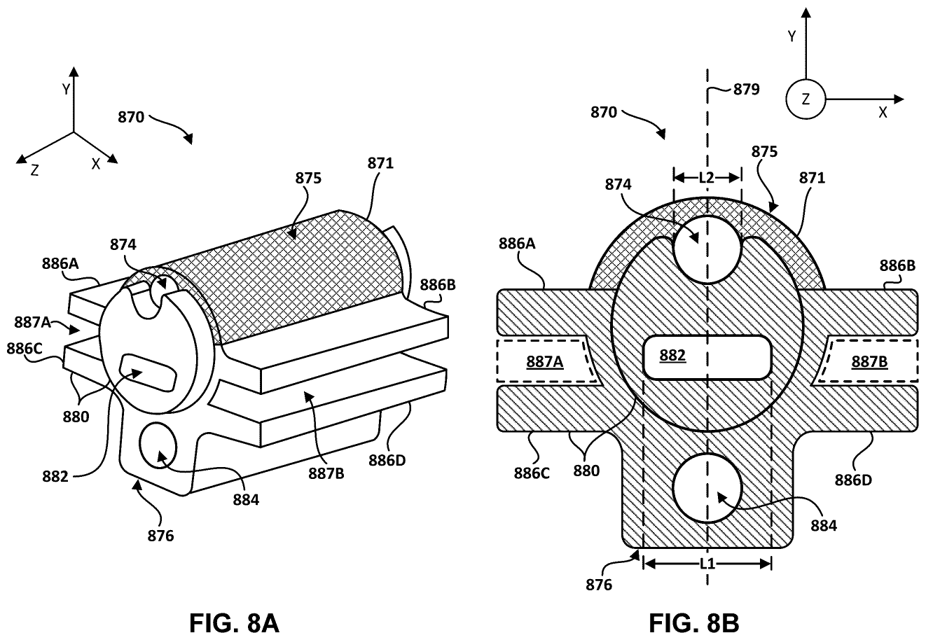

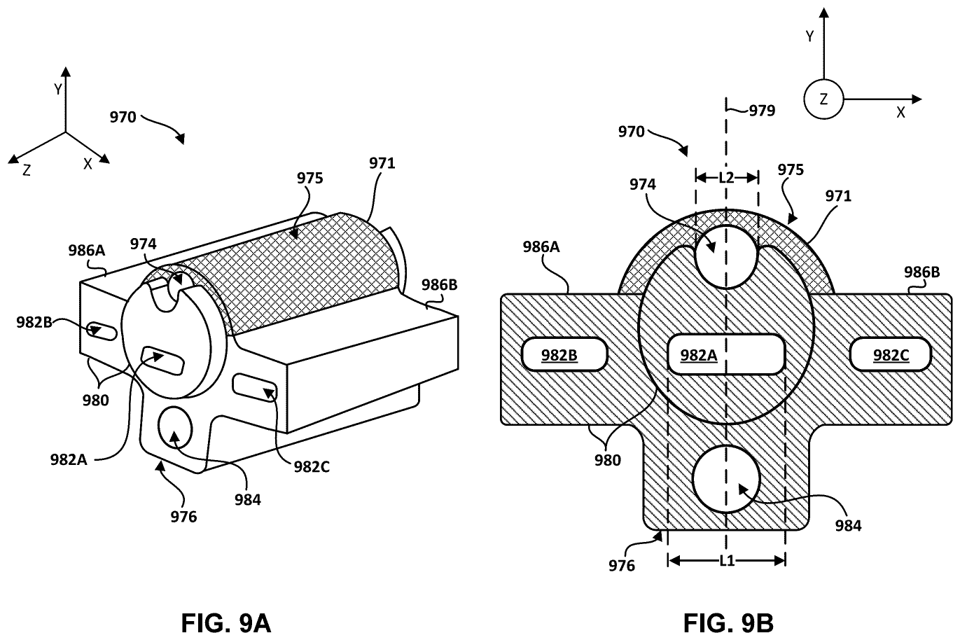

In the example of FIG. 3, expandable structure 190 includes structural features to facilitate mechanical coupling of electrode assemblies 170 to expandable structure 190 as well as orient electrode assemblies 170 with respect to expandable structure 190. As shown in FIG. 3, expandable structure 190 includes electrode attachment element 194A, electrode attachment element 194B, electrode attachment element 194C, electrode attachment element 194D, and electrode attachment element 194E, collectively referred to herein as electrode attachment elements 194. Each electrode attachment element 194A-194E is configured to mechanically couple to one or more of electrode assemblies 170 and orient one or more of orient electrode assemblies 170 with respect to expandable structure 190. For example, each electrode attachment element 194A-194E may be configured to mechanically couple to one or more of electrode assemblies 170 and orient electrode assemblies 170 such that an electrically conductive portion of each of electrode assemblies 170 faces (e.g., points) radially outward from expandable structure 190 (e.g., away from central longitudinal axis 191). In examples in which electrode assemblies 170 include an electrically insulative portion (e.g., such as discussed with respect to the examples of FIG. 8A and FIG. 8B as well as FIG. 9A and FIG. 9B), each electrode attachment element 194A-194E may be configured to orient electrode assemblies 170 such that the electrically insulative portion faces radially inward from expandable structure 190 (e.g., towards central longitudinal axis 191).

In some examples, each electrode attachment element 194A-194E is configured to minimize rotation (e.g., reduce or even eliminate rotation) of a given electrode of electrode assemblies 170 around one or more of struts 192 (e.g., by fixing an orientation of a given one of electrode assemblies 170 with respect to one or more of struts 192). For example, electrode attachment elements 194 can be configured to prevent an electrically conductive portion of electrode assemblies 170 from facing radially inward relative to expandable structure 190 (e.g., towards central longitudinal axis 191) and away from a blood vessel wall (e.g., away from a wall of jugular vein 13 as discussed with respect to FIG. 1).

Expandable structure 190 includes any suitable number of electrode attachment elements 194 configured to mechanically couple electrode assemblies 170 to expandable structure 190. As shown in the example of FIG. 3, expandable structure 190 includes five electrode attachment elements 194. However, expandable structure 190 can include any suitable number of electrode attachment elements 194 (e.g., one, two, three, four, five, six, seven, eight, nine, ten, twelve, fifteen, twenty, thirty, etc.). In some examples, expandable structure 190 includes more electrode attachment elements than electrode assemblies 170 that are eventually affixed to expandable structure 190, such that expandable structure 190 can be pre-fabricated, and a suitable number, configuration, and pattern of electrode assemblies 170 can subsequently be attached to expandable structure 190 depending on the end use of endovascular therapy system 100. Expandable structure 190 can include electrode attachment elements 194 at multiple circumferential positions around expandable structure 190 (e.g., about central longitudinal axis 191) and/or multiple longitudinal positions along expandable structure 190 (e.g., spaced apart along central longitudinal axis 191). Although referred to as circumferential positions, in some examples, expandable structure 190 is not circular in cross-section (the cross-section being taken in a direction orthogonal to central longitudinal axis 191). In such examples, the circumferential positions may still refer to the rotational position about central longitudinal axis 191).

Longitudinal spacing and/or circumferential spacing between electrode attachment elements can correspond to desired longitudinal spacing and/or circumferential spacing between electrode assemblies 170 for therapeutically effective endovascular stimulation and/or sensing. In some examples, circumferential spacing between adjacent electrode assemblies (e.g., once mechanically coupled to expandable structure 190 via electrode attachment elements 194 and with no other intervening electrode assemblies) is about 4 millimeters (mm) to about 8 mm around expandable structure 190. In some examples, an axial spacing and/or longitudinal spacing between adjacent electrode assemblies 170 is about 5 mm to about 10 mm. In some examples, a circumferential spacing between adjacent electrode assemblies 170 is less than an axial spacing and/or longitudinal spacing between adjacent electrode assemblies 170.

In some of these examples, not all the electrode attachment elements 194 of a given expandable structure 190 will be used to mechanically couple electrode assemblies 170 to expandable structure 190, but some electrode attachment elements 194 will remain unattached to electrode assemblies 170 after all of the desired number of electrode assemblies 170 are coupled to expandable structure 190.

Electrode attachment elements 194 can each include suitable configurations for being received by and/or otherwise mechanically coupling to one or more electrode assemblies of electrode assemblies 170. Similarly, electrode assemblies 170 can include a suitable configuration for mechanically receiving and/or otherwise mechanically coupling to electrode attachment elements 194. In some examples, as shown in the example of FIG. 3 with respect to electrode attachment element 194E, each electrode attachment element 194A-194E includes at least one projection 196 configured to be received by an electrode assembly 170. As shown in the example of FIG. 3, projection 196 for electrode attachment element 194E branches off of strut 192A. In some examples, projection 196 is configured to mechanically couple to mating portions of one or more of electrode assemblies 170.

In some examples, projection 196 is formed (e.g., integrally formed) as part of a unitary structure of expandable structure 190 along with interconnected struts 192. In some examples, expandable structure 190, including interconnected struts 192 and electrode attachment elements 194, is a single, continuous structure. For example, expandable structure 190, including interconnected struts 192 and/or electrode attachment elements 194, can be laser-cut from a single piece of material (e.g., nitinol, or another metallic material).

In other examples, electrode attachment elements 194 (e.g., which each can include the at least one projection 196) are formed separately from expandable structure 190, and subsequently attached to expandable structure 190. For example, electrode attachment elements 194 can be separately formed and attached to respective struts of struts 192 (e.g., electrode attachment element 194E including project 196 is formed separately from expandable structure 190 and subsequently attached to strut 192A of expandable structure 190). When formed separately from expandable structure 190, electrode attachment elements 194 can include a different material as expandable structure 190 (e.g., of struts 192), and/or different dimensions (e.g., cross-sectional dimensions) as struts 192.

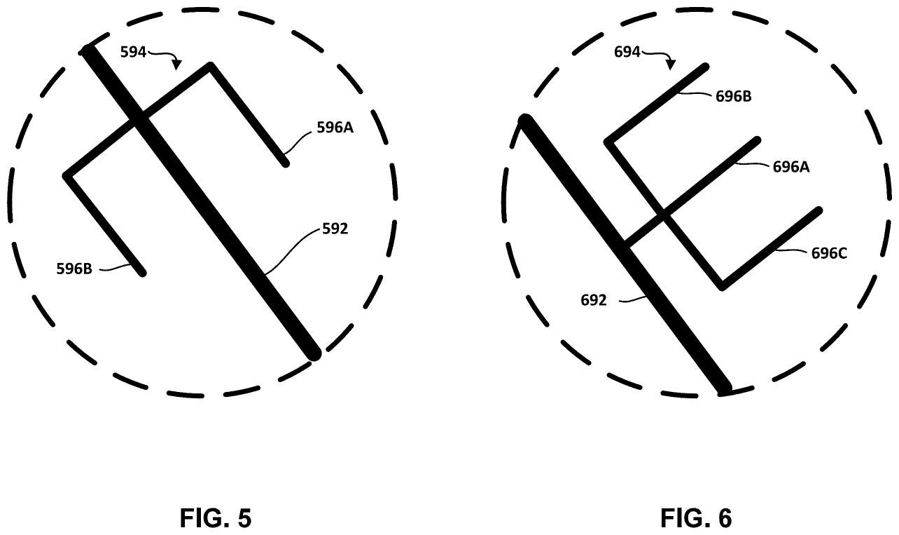

While the example of FIG. 3 shows electrode attachment elements 194 having one projection 196, electrode attachment elements 194 can include more than one projection 196 (e.g., two projections, three projections, four projections, five projections), such as is described with respect to the examples of FIG. 5 and FIG. 6. Multiple of projection 196 can facilitate mechanical connection of one or more electrode assemblies 170 to expandable structure 190 as described in this disclosure. In some examples, electrode attachment elements 194 include a corresponding number of projections 196 as respective mating structural features (e.g., holes and/or other structural features) on a respective electrode assembly 170. In some examples, each of the multiple projections 196 of a given one of electrode attachment elements 194 facilitate mechanical coupling of multiple electrode assemblies 170 to the given one of electrode attachment element electrode attachment elements 194.

Projection 196 of each electrode attachment elements 194A-194E can include any suitable configuration to facilitate mechanical coupling of electrode assemblies 170 to expandable structure 190. In the example of FIG. 3, electrode attachment elements 194A-194E are longitudinally spaced apart long expandable structure 190 (e.g., along central longitudinal axis 191) such that the respective projection 196 of each of electrode attachment elements 194A-194E branches off of different struts of struts 192A-192N. That is, projection 196 of each of electrode attachment elements 194 branch off of different struts of struts 192A-192N.

In some examples (such as shown and discussed with respect to the examples of FIG. 5 and FIG. 6), one or more (e.g., each) electrode attachment elements 194A-194E includes multiple projections. In some examples, the multiple projections of a given electrode attachment elements 194A-194E correspond to respective mating features of electrode assemblies 170.

In some examples, projection 196 of each electrode attachment elements 194 includes a suitable shape and size for mechanically engaging with a respective electrode assembly 170 to facilitate mechanically coupling of the respective electrode assembly 170 to expandable structure 190. As shown in the exploded view of electrode attachment element 194E, projection 196 branches off of strut 192A of expandable structure 190 such at least a portion of projection 196 extends away from strut 192A. In some examples, projection 196 includes a first portion that is parallel or substantially parallel (e.g., to the extent permitted by manufacturing tolerances) to strut 192A and a second portion that is perpendicular or substantially perpendicular (e.g., to the extent permitted by manufacturing tolerances) to strut 192A. For example, as shown in the example of FIG. 3, projection 196 forms an L-shape (e.g., a right-angle L-shape). In some examples, the portion of projection 196 received by one of electrode assemblies 170 is substantially parallel (e.g., to the extent permitted by manufacturing tolerances) to one of struts 192. By having a portion that is substantially parallel to one of struts 192, projection 196 can enable attachment of electrode assemblies 170 that additionally engage a portion one of struts 192 (e.g., as shown with respect to the example of electrode assembly 170D and electrode assembly 170B).

In other examples (e.g., including the example of FIG. 6), a portion of a projection configured to be received by one of electrode assemblies 170 is not parallel to one of struts 192. In some examples, a portion of a projection configured to be received by one of electrode assemblies 170 is perpendicular one of struts 192. In some examples, a portion of a projection configured to be received by one of electrode assemblies 170 is disposed at an angle (e.g., an angle between 0 degrees and 90 degrees) with respect to one of struts 192.

Projection 196 can have other suitable shapes, orientations, and positions relative to one or more struts 192. In some examples, projection 196 forms a curve. In some examples, projection 196 forms curved L-shape. In some examples, projection 196 branches off of a respective strut of struts 192 at a location between junctions of two of more of struts 192. In some examples, projection 196 branches off of a respective strut of struts 192 at a location proximate a junction of two of more of struts 192.

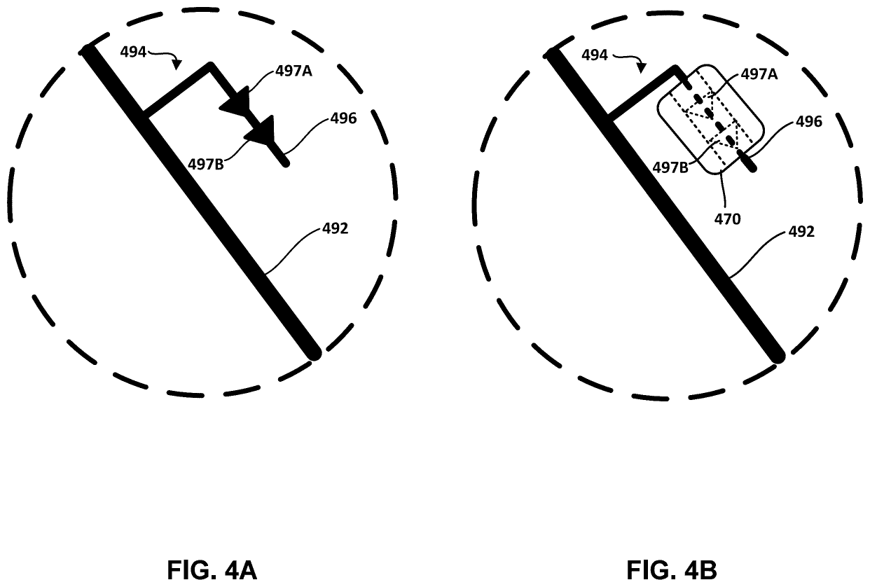

In some examples, projection 196 includes features to create an interference fit between projection 196 and one or more of electrode assemblies 170. For example, projection 196 can define at least a portion having a larger maximum dimension than an internal dimension of a respective mating portion of one of electrode assemblies 170. In some examples, such as is described with respect to FIG. 4A and FIG. 4B, projection 196 includes one or more barbs, wherein each of the one or more barbs are configured to facilitate an interference fit with one of electrode assemblies 170. Additionally or alternatively, another fixation means (e.g., adhesive, welding, or the like) can facilitate mechanical coupling of electrode attachment elements 194 (e.g. including projection 196) to one or more of electrode assemblies 170.

As described later in more detail, in some examples, some or all of electrode assemblies 170 are configured to receive one or more projections 196 of respective electrode attachment elements 194. For example, although an electrode assembly is not shown in conjunction with electrode attachment element 194E, an electrode assembly of electrode assemblies 170 can be configured to receive projection 196 via one or more structural (e.g., holes, apertures, recesses, etc.) defined by the respective electrode assembly of electrode assemblies 170. By receiving projection 196, one or more of electrode assemblies 170 can be configured to mechanically couple to expandable structure 190. In this way, each of electrode assemblies 170 are configured to mechanically couple to expandable structure 190 via respective electrode attachment elements 194.

In some examples, in conjunction with receiving respective projection 196 of electrode attachment elements 194, electrode assemblies 170 can additionally be fixedly coupled to the respective projection 196 via one or more of welding, adhesive, crimping, interference fit and/or another suitable method. In some examples, one or more of electrode assemblies 170 include another mechanical feature to facilitate mechanical coupling of electrode assemblies 170 to expandable structure 190. For example, each of electrode assemblies 170 can include one or more weld pads (e.g., to facilitate welding of electrode assemblies 170 to expandable structure 190), screw holes, thread holes (e.g., to facilitate mechanical connection via tie-down or threading), or the like to facilitate mechanical coupling of one or more of electrode assemblies 170 to expandable structure 190.

In some examples, such as the example of FIG. 3, electrode assemblies 170 are configured to receive projection 196 such that projection 196 extends entirely though electrode assemblies 170. As shown in FIG. 3, projection 196 extends away from each of electrode assemblies 170 (e.g., on opposite sides of each of electrode assemblies 170). As described more in relation to some later examples, each of electrode assemblies 170 can define holes (e.g., through holes) extending entire through each of electrode assemblies 170 for receiving projection 196. In other examples, each of electrode assemblies 170 define holes extending only partially through the body of each of electrode assemblies 170 (e.g., blind holes). In some examples, some holes of each of electrode assemblies 170 are blind holes, while other holes extend entirely through electrode assemblies 170 (e.g., holes for receiving projections 196 and/or conductor wires 166). In some examples, using blind holes can minimize and/or prevent fluid ingress such as to minimize and/or prevent interference with the mechanical connection between electrode assemblies 170 and expandable structure 190 and/or the electrical connection between electrode assemblies 170 and conductor wires 166, as well as to help prevent thrombus formation.

Endovascular therapy system 100 including electrode assemblies 170 mechanically coupled to expandable structure 190 may be configured to have a delivery configuration (e.g., compressed and/or relatively low-profile configuration) to facilitate delivery and/or placement into relatively narrow and/or tortuous vessels. Once proximate a target location, a clinician can cause expandable structure 190 to transform from the delivery configuration to a deployed configuration. In some examples, when expandable structure 190 is in a deployed configuration, at least a portion of each of electrode assemblies 170 (e.g., an outer surface of each of electrode assemblies 170) are flush or nearly flush with an outer surface of expandable structure 190. In some examples, electrode attachment elements 194 are configured to position electrode assemblies 170 such that at least one surface of each of electrode assemblies 170 is flush or nearly flush with the outer surface of expandable structure 190 when expandable structure 190 is in the delivery configuration.

In some examples, electrode assemblies 170 are configured to minimize impact to the ability of expandable structure 190 to transform between the delivery configuration (e.g., the compressed and/or relatively low-profile configuration) and the deployed (e.g., expanded) configuration. In some examples, electrode assemblies 170 are positioned, oriented, or otherwise configured with respect to portions of expandable structure 190 such that expandable structure 190 is able to transform between the delivery configuration and the deployed configuration with minimal or no interference (e.g., mechanical interference) from electrode assemblies 170. For example, electrode assemblies 170 can have suitable circumferential spacing and longitudinal (axial) spacing with respect to each other and/or adjacent structural features of expandable structure such as to not interfere with each other (e.g., adjacent electrode assemblies 170 do not interfere with each other) or other portions of expandable structure 190 during transformation of expandable structure 190 between the delivery configuration and the deployed configuration. In some examples, electrode assemblies 170 are positioned with respect to expandable structure 190 such as to minimize interference (e.g., mechanical interference) with one or more struts 192 (e.g., such that struts 192 may have relatively high freedom of movement with respect to each other so that expandable structure 190 is able to transform between the delivery configuration and the deployed configuration).

In the example of FIG. 3, system 100 includes a plurality of conductor wires 166 (shown individually as conductor wire 166A, conductor wire 166B, conductor wire 166C, and conductor wire 166D, but collectively referred to as conductor wires 166) configured to electrically connect electrode assemblies 170 to a medical device (e.g., medical device 14 in the example of FIG. 1). In some examples, each electrode assembly 170A-170D is configured to receive one or more conductor wires of conductor wires 166A-166D. For example, as described in more detail in relation to later examples, each of electrode assemblies 170A-170D can define one or more conductor holes configured to receive one or more conductor wires 166A-166D for electrically coupling respective electrode assemblies to a medical device (e.g., medical device 14 in the example of FIG. 1). The holes in each of electrode assemblies 170 for receiving and/or electrically connecting to one or more conductor wires 166 can extend partially or entirely though each of electrode assemblies 170. In some examples, to facilitate formation of this electrical connection between a conductor wire and respective electrode assembly, the one or more conductor holes includes an electrical contact that is electrically connected to the electrically conductive portion of the respective electrode assembly of electrode assemblies 170A-170D.

In some examples, one or more of electrode assemblies 170A-170D can define a conductor hole configured as a pass-through hole to facilitate electrical connection of the conductor wire to a second, different electrode assembly of one or more of electrode assemblies 170A-170D. For example, as shown in FIG. 3, electrode assembly 170D (which may be a proximal-most electrode) can define a conductor hole configured as a pass-through hole, such that a conductor wire (e.g., as shown, conductor wire 166C) that also connects to a more distal electrode assembly (e.g., electrode assembly 170C) passes through the conductor hole of electrode assembly 170D. In this way, in examples in which at least some of electrode assemblies 170 are not connected to a common conductor wire of conductor wires 166 (e.g., as shown in FIG. 3), some or all of electrode assemblies 170 may still include a conductor pass-through hole, such as for managing and/or routing conductor wires to other electrode assemblies of electrode assemblies 170. In this way, at least one of the electrode assemblies 170 can be configured to help hold the conductor wires relatively close to expandable structure 190 and out of the way of blood flowing through a lumen of expandable structure 190, which may help prevent thrombus formation. Additionally or alternatively, conductor holes being configured as pass through holes can enable multiple of electrode assemblies 170 to be electrically connected to a common conductor wire of conductor wires 166, such that more than one of electrode assemblies 170 can be controlled together (e.g., “shorted” together such that a medical device, such as medical device 14 of FIG. 1, can control electrode assemblies 170 simultaneously).

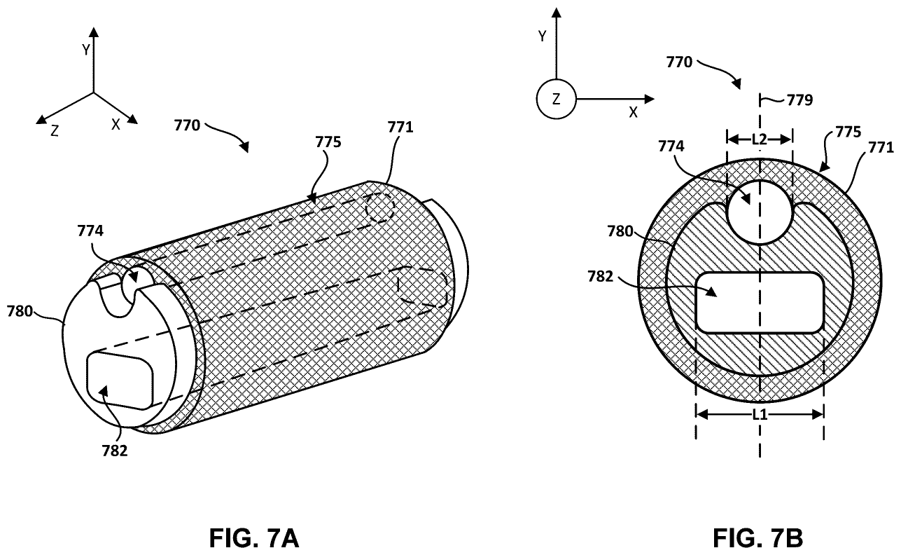

Electrode assemblies 170 can be fabricated using any suitable method, and some of all of electrode assemblies 170 can be formed according to the methods described herein. As described in connection with FIGS. 7A, 7B, 8A, 8B, 9A, and/or 9B, some or all of electrode assemblies 170 can include an electrically conductive portion formed from an electrically conductive material. In some examples, some or all of electrode assemblies 170 can include a combination of electrode assemblies having different form factors, structural features, and configurations (e.g., such as the different example configurations of electrode assemblies as discussed in connection with any of FIGS. 7A, 7B, 8A, 8B, 9A, and/or 9B). In some examples, certain types of electrode assemblies may be used for different purposes (e.g., some for delivering electrical stimulation therapy, some for sensing a patient parameter, and some for both delivering electrical stimulation therapy and for sensing a patient parameter).

Although FIG. 3 is described with respect to electrode assemblies 170 that are configured to deliver electrical stimulation therapy and/or sense electrical signals, endovascular therapy system 100 can additionally or alternatively include other types of therapy delivery elements and/or sensors. In some examples, endovascular therapy system 100 includes one or more ultrasound transducers, chemical delivery elements (e.g., fluid delivery elements and/or drug elution elements) which can be configured to be attached to expandable structure 190 using a similar method of attachment as electrode assemblies 170. In some examples, endovascular therapy system 100 additionally or alternatively includes one or more temperature sensors, pressure sensors, optical sensors, impedance sensors, chemical sensors, and/or other suitable types of sensors, which can be configured to be attached to expandable structure 190 using a similar method of attachment as electrode assemblies 170. In some examples, endovascular therapy system 100 includes one or more position indicators (e.g., radiopaque elements integrated into or carried by at least a portion of medical lead 160 and/or expandable structure 190).

FIG. 4A and FIG. 4B illustrate an example electrode attachment element 494, which is an example of any of electrode attachment elements 194A-194E of FIG. 3. Electrode attachment element 494 includes a projection 496 which may be similar to projection 196 of FIG. 3 except as discussed herein. Projection 496 branches off of a strut 492, which is an example of any of struts 192 (e.g., strut 192A) of expandable element 190 of FIG. 3. As shown in in the example of FIG. 4B, an example electrode assembly 470, which may be an example of any of the electrode assemblies discussed herein (e.g., electrode assemblies 17, electrode assemblies 170, or any of the other electrode assemblies discussed herein) is mechanically coupled to electrode attachment element 494.

In the example of FIG. 4A and FIG. 4B, projection 496 includes a first barb 497A and a second barb 497B (collectively referred to herein as barbs 497). While the example of FIG. 4A and FIG. 4B illustrates projection 496 as having two barbs 497, projection 496 can have any suitable number of barbs 497 (e.g., one barb, three barbs, four barbs, five barbs, or another suitable number of barbs). Barbs 497 can be disposed at respective, spaced-apart locations along projection 496, such as at respective locations along a portion of projection 496 that is parallel to strut 492. Further, each of the barbs 497 can rotationally extend fully or partially around the respective projection 496.

In some examples, one or more of barbs 497 is configured to facilitate mechanical coupling of electrode assembly 470 to electrode attachment element 494, which is an example of any of electrode attachment elements 194A-194E of FIG. 3, or any of the other electrode attachment elements discussed herein. For example, one or more of barbs 497 can be configured to create an interference fit between electrode assembly 470 and a portion of respective electrode attachment element 494 (e.g., an interference fit between the electrode assembly and projection 496 and/or barbs 497). In some examples, as described more fully below, electrode assembly 470 can define one or more fixation holes configured to receive projection 496 and/or one or more of barbs 497. In some examples, a portion of electrode assembly 470 can be configured to deform to accommodate barbs 497, such as to create an interference fit between barbs 497 and a portion (e.g., the fixation hole) of electrode assembly 470. The interference fit can enable electrode assembly 470 to be mechanically coupled to projection 496 of electrode attachment element 494. In some examples, electrode assembly 470 is additionally or alternatively mechanically coupled to electrode attachment element 494 via welding, adhesive (e.g., to bond a surface of electrode assembly 470 to a surface of one or more of projection 496 and/or strut 492), or the like.

Other mating and/or complementary configurations of electrode attachment element 494, including barbs 497, and electrode assembly 470 are contemplated. In some examples, barbs 497 are configured to reversibly deform (e.g., deform radially inward toward projection 496) when projection 496 receives electrode assembly 470. In some examples, electrode assembly 470 includes one or more features configured to mate with barbs 497. For example, electrode assembly 470 can define internal pockets or notches configured to receive barbs 497 (e.g., to facilitate a snap-fit with barbs 497). In some examples, barbs 497 are configured to deform when projection 496 receives electrode assembly 470, and subsequently expand radially outward when barbs 497 are aligned with the pockets or notches of electrode assembly 470.

FIG. 5 illustrates and example electrode attachment element 594, which is another example of any of electrode attachment elements 194A-194E of FIG. 3. Electrode attachment element 594 includes a first projection 596A and a second projection 596B (collectively referred to herein as projections 596). Each of first projection 596A and second projection 596B branch off of a strut 592, which may be an example of any of struts 192 (e.g., strut 192A) of expandable element 190 of FIG. 3. In some examples, each of first projection 596A and/or second projection 596B are configured to mechanically couple to mating portions of one or more of electrode assemblies (e.g., any of electrode assemblies 170A-170D in the example of FIG. 3, or any of the other electrode assemblies discussed herein).

As shown in the example of FIG. 5, first projection 596A and second projection 596B branch off of strut 592 (which may be a strut of an expandable structure, such as strut 192A of expandable structure 190 of FIG. 3) in opposite directions from strut 592, such that first projection 596A and second projection 596B are on an opposite sides of strut 592. Said another way, first projection 596A extends away from a first side of strut 592 and second projection 596B extends away from a second side of strut 592 opposite the first side. In some examples, first projection 596A and second projection 596B are symmetrical (e.g., reflectionally symmetric) about strut 592. In other examples, first projection 596A and second projection 596B are asymmetrically positioned relative to strut 592.

In some examples, each of first projection 596A and a second projection 596B are configured to mechanically couple to the same electrode assembly. In other examples, each of first projection 596A and a second projection 596B are configured to mechanically couple to separate electrode assemblies. For example, a first electrode assembly (e.g., such as electrode assembly 170A of FIG. 3) can be positioned on and carried by first projection 596A, and a second, different electrode assembly (e.g., such as a second electrode assembly 170A of FIG. 3) can be positioned on and carried by second projection 596B. In this way, electrode attachment element 594 as shown in FIG. 5 can enable assembly of an endovascular therapy system (e.g., endovascular therapy system 100) with selectable electrode density at a given longitudinal or circumferential location corresponding to each electrode attachment element by enabling attachment of more (e.g., additional) or less (e.g., fewer) electrode assemblies to each electrode attachment element.

FIG. 6 illustrates an example electrode attachment element 694, which is another example of any of electrode attachment elements 194A-194E of FIG. 3. Electrode attachment element 694 includes a first projection 696A, a second projection 696B, and a third projection 696C (collectively referred to herein as projections 696). Each of first projection 696A, second projection 696B, and third projection 696C branch off of a strut 692, which is an example of any of struts 192 (e.g., strut 192A) of expandable element 190 of FIG. 3. In some examples, each of first projection 696A, second projection 696B, and third projection 696C are configured to mechanically couple to mating portions of one or more of electrode assemblies (e.g., any of electrode assemblies 170A-170D of FIG. 3).

In some examples, the portion of each of each of first projection 696A, second projection 696B, and third projection 696C configured to be received by a one or more of electrode assemblies (e.g., any of electrode assemblies 170A-170D) is not parallel to strut 692. Rather, as shown in the example of FIG. 6, the portion of each of each of first projection 696A, second projection 696B, and third projection 696C configured to be received by a one or more of electrode assemblies (e.g., any of electrode assemblies 170A-170D) is substantially perpendicular (e.g., perpendicular or nearly perpendicular to the extent permitted by manufacturing tolerances) to strut 692.

As shown in the example of FIG. 6, first projection 696A, second projection 696B, and third projection 696C branch off of strut 692 (which is an example of a strut of an expandable structure, such as strut 192A of expandable structure 190 of FIG. 3) in a common direction away from strut 692, such that first projection 696A, second projection 696B, and third projection 696C are on a common side of strut 692. In some examples, first projection 696A, second projection 696B, and third projection 696C of electrode attachment element 694 form a trident shape. In some examples, as shown in the example of FIG. 6, each of first projection 696A, second projection 696B, and third projection 696C include at least a portion that are parallel to each other, with first projection 696A between second projection 696B and third projection 696C.

In some examples, a spacing and orientation of each of first projection 696A, second projection 696B, and third projection 696C with relation to each other corresponds to mating features of an electrode assembly (e.g., one or more of electrode assemblies 170 of FIG. 3, or other electrode assemblies discussed throughout this disclosure). For example, as discussed with respect to FIG. 9A and FIG. 9B, an electrode assembly can include respective holes (e.g., spaced apart fixation holes) configured to respectively receive first projection 696A, second projection 696B, and third projection 696C.

In some examples, each of first projection 696A, second projection 696B, and third projection 696C are configured to mechanically couple to separate electrode assemblies. In this way, as discussed in relation to FIG. 5, electrode attachment element 694 as shown in FIG. 6 can additionally or alternatively enable assembly of an endovascular therapy system (e.g., endovascular therapy system 100) with selectable electrode density at a given longitudinal or circumferential location corresponding to each electrode attachment element by enabling attachment of more (e.g., additional) or less (e.g., fewer) electrode assemblies to each electrode attachment element.

FIGS. 7A, 7B, 8A, 8B, 9A, and 9B illustrate various examples of electrode assemblies, which are examples of electrode assemblies suitable for use as electrode assemblies 17 and/or electrode assemblies 170 described in connection with the systems of FIG. 1 and FIG. 3 respectively. In particular, each of FIGS. 7A, 7B, 8A, 8B, 9A, and 9B, illustrate example electrode assemblies configured to receive at least one projection of an electrode attachment element and configured to receive at least one conductor wire via a first conductor hole. FIGS. 8A, 8B, 9A, and 9B illustrate electrode assemblies that additionally define a second conductor hole (e.g., which may be configured as a pass-through hole to facilitate connection of a second conductor wire to a different electrode assembly). FIG. 8A and FIG. 8B illustrate an electrode assembly that is additionally configured to receive and/or engage with a strut of an expandable structure (e.g., one of struts 192 of expandable structure 190). FIG. 9A and FIG. 9B illustrate an electrode assembly that is additionally configured to receive at least a first projection and a second projection of an electrode attachment element.

FIG. 7A and FIG. 7B illustrate a perspective view and a side view, respectively, of an electrode assembly 770, which is an example of one of electrode assemblies 17 of FIG. 1 and/or one of electrode assemblies 170 of FIG. 3 (e.g., such as electrode assembly 170A and/or electrode assembly 170C) and/or electrode assembly 470 of FIG. 4. Electrode assembly 770 includes an electrically conductive portion 771 that defines an electrically conductive surface 775 (which may be referred to as a first surface). Electrode assembly 770 includes an electrically insulative portion 780 configured to electrically insulate at least a portion of electrically conductive portion 771 from another structure (e.g., a portion of lead 160, expandable structure 190 of FIG. 3, an anatomical structure, biological matter, and/or the like).

For illustrative purposes, some elements described in FIG. 3 will be referenced herein with respect to electrode assembly 770 (e.g., as corresponding mating structural features), however it should be understood that electrode assembly 770 is only one example of electrode assemblies 170 of FIG. 3, and that other types of electrode assemblies can additionally or alternatively be used in conjunction with the elements described in connection with FIG. 3. For example, electrode attachment element 194E, including projection 196, as well as strut 192A of interconnected struts 192 of FIG. 3 will be referenced to illustrate how electrode assembly 770, shown in FIG. 7A and FIG. 7B, can be configured to attach to expandable structure 190 of FIG. 3.

Electrode assembly 770 is configured to mechanically couple to another structure (e.g., a medical lead or expandable structure, such as medical lead 160 and/or expandable structure 190 of FIG. 3). In some examples, electrode assembly 770 includes one or more structural features configured to facilitate mechanical coupling of electrode assembly 770 to another structure (e.g., a medical lead or expandable structure, such as medical lead 160 and/or expandable structure 190 of FIG. 3). For example, electrically insulative portion 780 of electrode assembly 770 is configured to facilitate mechanical coupling of electrode assembly 770 to expandable structure 190. Electrically insulative portion 780 can also be configured to electrically insulate one or more electrically conductive portions of electrode assembly 770 (e.g., electrically conductive portion 771) from portions of expandable structure 190. In this way, electrically insulative portion 780 can be configured to both facilitate mechanical coupling of electrode assembly 770 to expandable structure 190 as well facilitate electrical insulation of at least a portion of electrode assembly 770 from another structure (e.g., at least a portion of expandable structure 190). This dual purpose can reduce or even eliminate the need to provide separate features and/or separate components to serve each of these mechanical coupling and electrically insulating functions. This dual purpose may shorten the time needed for manufacturing and/or simplify manufacturing and/or assembly of electrode assembly 770. Further, this dual purpose served by electrically insulative portion 780 can also help maintain a relatively low profile electrode assembly 770 and endovascular device including electrode assembly 770. A relatively low profile electrode assembly 770 can be particularly advantageous when used in vasculature of a patient. For example, the low profile can facilitate easier navigation to a target site in vasculature of a patient as well as minimize adverse impacts to blood flow in the vasculature by the endovascular device that includes the relatively low profile electrode assembly 770.

Electrically conductive portion 771 can define any suitable shape. In some examples, electrically conductive portion 771 defines a tubular (e.g., cylindrical) shape, ring-like shape, or the like. In some examples, electrically conductive portion 771 includes a hollow cylinder. In some examples, as discussed herein, at least a sub-portion electrically conductive portion 771 is covered by an electrically insulative material (e.g., electrically insulative material 780). For example, as shown in the example FIG. 7A and FIG. 7B, electrically insulative portion 780 covers at least an interior portion (e.g., radially inward portion when electrode assembly 770 is connected to expandable structure 190) of electrically conductive portion 771. In examples in which electrically conductive portion 771 defines a hollow cylinder shape, electrically insulative portion 780 covers at least a portion of the interior and/or the exterior of the hollow cylinder shape.

Electrically conductive portion 771 (which can include a cylindrical shape) can be formed using any suitable technique. In some examples, the electrically conductive portion 771 is formed via a machining or stamping process.

As shown in the example of FIG. 7A and FIG. 7B, electrode assembly 770 (e.g., including the exposed radially outward portion of electrically conductive portion 771) defines electrically conductive surface 775. In some examples, electrically conductive surface 775 is configured to transmit and/or receive electrical signals, such as in examples in which electrode assembly 770 is positioned on expandable structure 190 of endovascular therapy system 100 (e.g., in which endovascular therapy system 100 is configured for endovascular stimulation therapy and/or sensing a patient parameter). As shown in the example of FIG. 7A and FIG. 7B, at least a portion (e.g., a radially outward portion) electrically conductive surface 775 is not covered by an electrically insulative material (e.g., not covered by electrically insulative portion 780 of electrode assembly 770).

In some examples, electrically conductive surface 775 is configured to be placed proximate (e.g., in apposition with) a vessel wall, such as to maintain contact against a vessel wall for electrical stimulation therapy and/or sensing. When affixed to expandable structure 190, electrically conductive surface 775 is configured to face radially outward from expandable structure 190, such as in a direction radially outward from central longitudinal axis 191 of expandable structure 190 of FIG. 3. In the example of FIG. 7A and FIG. 7B, electrically conductive surface 775 faces at least in the positive y-axis direction according to the orthogonal x-y-z axes shown in FIG. 7A and FIG. 7B. However, as shown in the example of FIG. 7A and FIG. 7B, electrically conductive surface 775 faces in additional directions. In the example of FIG. 7A and FIG. 7B, electrically conductive surface 775 faces radially outward in all directions radially outward from a longitudinal axis of electrode assembly 770, such as an axis parallel to the z-axis according to the orthogonal x-y-x axes shown in the example of FIG. 7A and FIG. 7B.

In the example of FIG. 7A and FIG. 7B, electrically conductive surface 775 is a curved surface (e.g., which may correspond to a cylindrical-shape of electrically conductive portion 771 and/or circular or oval-shaped cross section of electrically conductive portion 771). However, in other examples, electrically conductive surface 775 is flat, concave, irregular, or defines another suitable shape.

In some examples, electrically conductive portion 771 of electrode assembly 770 includes a suitable electrically conductive material configured for transmitting and/or receiving electrical signals. In some examples, electrically conductive portion 771 of electrode assembly 770 includes one or more of titanium (Ti), tantalum (Ta), Tin (Sn), and/or a suitable combination thereof (e.g., TiTaSn and/or similar alloys). In some examples, electrode assembly 770 includes one or more of platinum (Pt) and/or Iridium (Ir) and/or a suitable combination thereof (e.g., PtIr and/or similar alloys). However, electrically conductive portion 771 of electrode assembly 770 can include other electrically conductive materials and/or combinations of materials.

In some examples, electrically conductive surface 775 includes a coating or other surface treatment to facilitate transmitting and/or receiving electrical signals. In some examples, electrically conductive surface 775 includes a coating comprising one or more of Titanium Nitride (TiN), Iridium Oxide (IrOx), another suitable material, or a combination thereof. In some examples, electrically conductive surface 775 additionally or alternatively includes a surface treatment, such as laser texturing to facilitate transmitting and/or receiving electrical signals. The surface treatment (e.g., laser texturing or mechanical texturing) can help effectively increase the electrically conductive surface area of electrically conductive surface 775 for a given footprint of electrically conductive portion 771 of electrode assembly 770.

As shown in the examples of FIG. 7A and FIG. 7B, electrode assembly 770 includes electrically insulative portion 780. In some examples, as shown in FIG. 7A and FIG. 7B, electrically insulative portion 780 is positioned in and occupies a radially inward portion of electrode assembly 770 (e.g., as compared to electrically conductive portion 771). For example, electrically insulative portion 780 is positioned in and occupies an area of electrode assembly 770 closest to a central longitudinal axis of electrode assembly 770 that extends in a direction parallel to the z-axis.

Electrically insulative portion 780 includes a suitable material or combination of materials configured for electrically insulating at least a portion of electrode assembly 770 from another structure (e.g., one or more portions of expandable structure 190 including projection 196, as well as other structures including conductor wires 166, anatomical features, biological tissue, etc.). In some examples, electrically insulative portion 780 includes a polycarbonate urethane (PCU), silicone, polypropylene, polyethylene, polystyrene, or polyetheretherketone (PEEK), another polymer, another suitable electrically insulative material (e.g., non-polymer), and/or any suitable combination or sub-combination thereof.

Electrically insulative portion 780 is configured to be formed from any suitable processes or combination of processes. In some examples, electrically insulative portion 780 is formed from an injection molding process. In some examples, electrically insulative portion 780 of electrode assembly 770 includes an injection molded polymer. In some examples, electrically insulative portion 780 is configured to be injection molded (e.g., insert molded) around electrically conductive portion 771 (e.g., such that electrically conductive portion 771 can be placed into an injection molding mold, and electrically insulative portion 780 is formed around at least a portion of electrically conductive portion 771). Using a suitable injection molding process for forming electrically insulative portion 780 may enable electrode assembly 770 to define relatively complex geometries and/or relatively complex overall shape while allowing electrically conductive portion 771 to be a relatively simply geometry (e.g., a relatively simply geometrical shape such as a cylinder or disk). As creating complex geometries with injection molding of suitable polymers can be relatively easier than creating complex geometries of electrically conductive materials (e.g., PtIr or other electrically conductive materials discussed herein), the configurations of electrode assemblies discussed herein can facilitate relatively easier and/or simpler manufacturing and/or assembly of such electrode assemblies.

In other examples, electrically insulative portion 780 is configured to be formed (e.g., by a suitable forming process such as injection molding) and subsequently coupled with electrically conductive portion 771. For example, electrically insulative portion 780 can be formed via a suitable process and subsequently coupled with (e.g., via press fit, adhesive, or another suitable mechanical connection) with electrically conductive portion 771.

Electrode assembly 770 is configured to receive one or more structural features of a medical lead and/or other structures (e.g., features of medical lead 160 and/or expandable structure 190 of FIG. 3), such as for mechanically coupling one or more of electrode assembly 770 to medical lead 160 and/or expandable structure 190. The mechanical coupling of one or more of electrode assembly 770 to expandable structure 190 according to the techniques of this disclosure can help orient electrode assembly 770 with respect to the vasculature such that at least a portion of electrically conductive surface 775 faces radially outward and towards a vessel wall when expandable structure 190 with electrode assembly 770 are placed and deployed within the vasculature.

In some examples, electrode assembly 770 includes one or more structural features configured to receive portions of an expandable structure (e.g., expandable structure 190 of FIG. 3). In the example of FIG. 7A and FIG. 7B, electrode assembly 770 (e.g., electrically insulative portion 780) defines a fixation hole 782, which is configured to receive respective a portion of electrode attachment elements 194 of FIG. 3. For example, in some examples, fixation hole 782 is configured to receive projection 196 of electrode attachment element 194E.

Fixation hole 782 may be sized, oriented, and otherwise configured for receiving mating portions of expandable structure 190 (e.g., projection 196 of electrode attachment element 194E). In some examples, fixation hole 782 defines a maximum dimension L1 (which may be an internal maximum dimension). Maximum dimension L1 may be a diameter in examples in which fixation hole 782 defines a circular or approximately circular shape (e.g., a circular or approximately circular cross-section in a plane defined by the x- and y-axes).

In some examples, electrically insulative portion 780, which may be configured to receive a portion of an electrode attachment element 194E including projection 196, is configured to deform to create an interference fit between electrically insulative portion 780 and a portion of electrode attachment element 194E, such as projection 196. As discussed in relation to some previous examples (e.g., FIG. 4A and FIG. 4B), a projection (e.g., projection 496) can include features (e.g., barbs 497) that facilitate the interference fit between the projection and electrically insulative portion 780. In some examples, as discussed above with respect to FIG. FIG. 4A and FIG. 4B, projection 496 (including barbs 497) can be configured to mate with electrically insulative portion 780 (e.g., one or more internal notches or pockets defined by electrically insulative portion 780, such as to create a snap fit). In some examples, maximum dimension L1 changes upon insertion of a respective mating feature (e.g., projection 196 of electrode attachment element 194E).

In some examples, maximum dimension L1 corresponds to a maximum outer dimension of a respective mating feature (e.g., projection 196 of electrode attachment element 194E). In some examples, maximum dimension L1 is equal to or slightly larger than the respective mating feature (e.g., projection 196). In other examples, maximum dimension L1 is equal to or slightly smaller than the respective mating feature (e.g., projection 196), such as in examples in which fixation hole 782 is configured to create an interference fit with the respective mating feature (e.g., projection 196).

In some examples, as shown in the example of FIG. 7A and FIG. 7B, fixation hole 782 is a through hole and extends entirely through electrode assembly 770 (e.g., extends entirely through electrically insulative portion 780 in a direction along the z-axis). In other examples, fixation hole 782 extends only partially through electrode assembly 770 (e.g., only partially through electrically insulative portion 780 in a direction along the z-axis), such that fixation hole 782 is a blind hole. In some examples, using blind holes can minimize and/or prevent fluid ingress such as to minimize and/or prevent interference with the mechanical connection between electrode assemblies 770 and expandable structure 190, as well as help prevent thrombus formation.

In some examples, electrode assembly 770 is configured to electrically connect to one or more conductor wires, such as to facilitate electrical communication of electrode assembly 770 (e.g., electrically conductive portion 771) with a medical device (e.g., medical device 14 of FIG. 1). In some examples, a portion of electrode assembly 770 (e.g., a portion of one or more of electrically conductive portion 771 and electrically insulative portion 780) defines a conductor hole 774. In some examples, conductor hole 774 is configured to receive a conductor wire (e.g., one or more of conductor wires 166 of FIG. 3) for electrically coupling electrode assembly 770 to a medical device. One or more of conductor wires 166 can be mechanically coupled to electrode assembly 770 (e.g., once one of conductor wires is positioned in conductor hole 774) via a suitable mechanical connection (e.g., welding, adhesive, interference fit in conductor hole 774, or the like). In some examples, conductor hole 774 includes an electrical contact electrically connected to electrically conductive portion 771 of electrode assembly 770. Thus, when a conductor wire 166 is introduced into conductor hole 771 and electrically connected to the electrical contact, the conductor wire is electrically connected to electrically conductive portion 771.

In some examples, conductor hole 774 is a through hole and extends entirely through electrode assembly 770 (e.g., extending entirely through electrode assembly 770 in a direction along the z-axis). In other examples, conductor hole 774 extends only partially through electrode assembly 770 (e.g., only partially through electrode assembly 770 in a direction along the z-axis), such that conductor hole 774 is a blind hole. In some examples, using blind holes can minimize and/or prevent fluid ingress such as to minimize and/or prevent interference with the electrical connection between electrode assemblies 170 and conductor wires 166 and help prevent thrombus formation. In some examples, conductor hole 774 is formed from both electrically conductive portion 771 and electrically insulative portion 780 of electrode assembly 770. In other examples, conductor hole 774 is formed from only one of electrically conductive portion 771 or electrically insulative portion 780.

In some examples, conductor hole 774 is sized to accommodate one conductor wire (e.g., of conductor wires 166). In some examples, conductor hole 774 defines a maximum dimension L2 (which may be an internal dimension of conductor hole 774). In some examples, maximum dimension L2 is slightly larger than a maximum cross-sectional dimension (e.g., a width) of one conductor wire (e.g., of conductor wires 166). In examples in which conductor hole 774 defines a circular or approximately circular shape, maximum dimension L2 is a diameter.

In some examples, conductor hole 774 is configured as a pass-through hole for one or more conductor wires (e.g., conductor wires 166 of FIG. 3). Conductor hole 774 being configured as a pass-through hole can facilitate electrical connection of the conductor wire (e.g., one or more of conductor wires 166) to a second, different electrode assembly. For example, one or more of conductor wires 166 may be fed through conductor hole 774 and electrically connected to a different (e.g., more distal) electrode. In this way, multiple of electrode assemblies can be electrically connected to a common conductor wire of conductor wires 166 (e.g., “shorted together”), such that more than electrode assemblies can be controlled together (e.g., by a medical device, such as medical device 14 of FIG. 1).

In some examples, electrode assembly 770 is sized, shaped, or otherwise configured to facilitate relatively faster and/or relatively easier fabrication of one or more of electrode assembly 770 and/or assembly with other portions of a medical device system (e.g., expandable structure 190). In some examples, electrode assembly 770 is sized, shaped, or otherwise configured to facilitate relatively easy attachment to expandable structure 190 (e.g., such as to reduce assembly errors, poka-yoke, or the like). For example, as explained below, electrode assembly can include symmetry and/or redundant features that enable electrode assembly 770 to be mechanically coupled to expandable structure 190 in more than one orientation, and electrode assembly 770 may function similarly or the same in each of the one or more orientations.

In some examples, at least a portion of electrode assembly 770 is symmetric (e.g., reflectionally symmetric). As shown in FIG. 7B, electrode assembly 770 is symmetric (e.g., reflectionally symmetric) about (e.g., relative to) a medial plane 779 intersecting a midpoint on an electrode assembly face (e.g., the face of electrode assembly 770 facing in the positive z-axis direction in the example of FIG. 7A and FIG. 7B). In the example of FIG. 7B, medial plane 779 is parallel to the y-axis and extends through electrode assembly 770 in the z-axis direction. This reflectional symmetry may enable electrode assembly 770 to be attached to expandable structure 190 in at least two different orientations. For example, electrode assembly 770 can be attached to projection 196 of electrode attachment element 194E with either the face of electrode assembly 770 (e.g., either face of insulative portion 780) in the positive z-axis direction or the negative z-axis direction as the leading face. Symmetry of electrode assembly 770 may also facilitate balance of electrode assembly 770 when fixed to one of electrode attachment elements 194.

In some examples, as shown in FIG. 7B, conductor hole 774 is symmetric (e.g., reflectionally symmetric) about medial plane 779. In some examples, conductor hole 774 intersects medial plane 779. While the example of FIG. 7A and FIG. 7B depict electrode assembly 770 as symmetric about medial plane 779, other configurations (including other symmetrical configurations) are contemplated.

In other examples, electrode assembly 770, including one or more structural features of electrode assembly 770, is asymmetric (e.g., asymmetric about medial plane 779). In some examples, conductor hole 774 is not symmetric about medial plane 779 and/or does not intersect medial plane 779. In some examples, electrically conductive surface 775 is asymmetric relative to medial plane 779 (e.g., electrically conductive surface 775 extends relatively more in the positive x-axis direction or the negative x-axis direction in the example of FIG. 7A and FIG. 7B). In some examples, asymmetry of electrode assembly 770 (including electrically conductive surface 775) facilitates a relative larger conductive surface (e.g., electrically conductive surface 775) as compared to other configurations.

FIG. 8A and FIG. 8B illustrate a perspective view and a side view, respectively, of an electrode assembly 870, which is another example of one of electrode assemblies 17 of FIG. 1 and/or one of electrode assemblies 170 of FIG. 3 (e.g., such as electrode assembly 170B and/or electrode assembly 170D). Electrode assembly 870 includes an electrically conductive portion 871 that defines an electrically conductive surface 875 (which may be referred to as a first surface). Electrode assembly 870 includes an electrically insulative portion 880 configured to electrically insulate at least a portion of electrically conductive portion 871 from another structure (e.g., a portion of lead 160, expandable structure 190 of FIG. 3, an anatomical structure, biological matter, and/or the like). Electrically insulative portion 880 defines at least one electrically insulative surface 876 (which may be referred to as a second surface), which may be positioned opposite electrically conductive surface 875, as described more fully herein.

For illustrative purposes, some elements described in FIG. 3 will be referenced herein with respect to electrode assembly 870 (e.g., as corresponding mating structural features), however it should be understood that electrode assembly 870 is only one example of electrode assemblies 170 of FIG. 3, and that other types of electrode assemblies can additionally or alternatively be used in conjunction with the elements described in connection with FIG. 3. For example, electrode attachment element 194E, including projection 196, as well as strut 192A of interconnected struts 192 of FIG. 3 will be referenced to illustrate how electrode assembly 870, shown in FIG. 8A and FIG. 8B, can be configured to attach to expandable structure 190 of FIG. 3.

Electrode assembly 870 is configured to mechanically couple to another structure (e.g., a medical lead or expandable structure, such as medical lead 160 and/or expandable structure 190 of FIG. 3). In some examples, electrode assembly 870 includes one or more structural features configured to facilitate mechanical coupling of electrode assembly 870 to another structure (e.g., a medical lead or expandable structure, such as medical lead 160 and/or expandable structure 190 of FIG. 3). For example, electrically insulative portion 880 of electrode assembly 870 is configured to facilitate mechanical coupling of electrode assembly 870 to expandable structure 190. Electrically insulative portion 880 can also be configured to electrically insulate one or more electrically conductive portions of electrode assembly 870 (e.g., electrically conductive portion 871) from portions of expandable structure 190. In this way, electrically insulative portion 880 can be configured to both facilitate mechanical coupling of electrode assembly 870 to expandable structure 190 as well facilitate electrical insulation of at least a portion of electrode assembly 870 from another structure (e.g., at least a portion of expandable structure 190). This dual purpose can reduce or even eliminate the need to provide separate features and/or separate components to serve each of these mechanical coupling and electrically insulating functions. This dual purpose may shorten the time needed for manufacturing and/or simplify manufacturing and/or assembly of electrode assembly 870. Further, this dual purpose served by electrically insulative portion 880 can also help maintain a relatively low profile electrode assembly 870 and endovascular device including electrode assembly 870. A relatively low profile electrode assembly 870 can be particularly advantageous when used in vasculature of a patient. For example, the low profile can facilitate easier navigation to a target site in vasculature of a patient as well as minimize adverse impacts to blood flow in the vasculature by the endovascular device that includes the relatively low profile electrode assembly 870.

Prior to assembly into the final form factor of electrode assembly 870, electrically conductive portion 871 can define any suitable shape. In some examples, electrically conductive portion 871 defines a tubular (e.g., cylindrical) shape, a half-cylinder shape, ring-like shape, or the like. In some examples, electrically conductive portion 871 includes a hollow cylinder. In some examples, as discussed herein, at least a sub-portion electrically conductive portion 871 is configured to be covered by an electrically insulative material (e.g., electrically insulative material 880). In some examples, the electrically insulative material (e.g., electrically insulative material 880) covers at least a portion of electrically conductive portion 871. For example, as shown in the example FIG. 8A and FIG. 8B, electrically insulative portion 880 covers at least an interior portion (e.g., radially inward portion) as well as an exterior portion (e.g., radially outward portion) of electrically conductive portion 871. In examples in which electrically conductive portion 871 defines a hollow cylinder shape, electrically insulative portion 880 covers at least a portion of the interior and the exterior of the hollow cylinder shape.

Electrically conductive portion 871 (which can include a cylindrical shape) can be formed using any suitable technique. In some examples, the electrically conductive portion 871 is formed via a machining or stamping process.

As shown in the example of FIG. 8A and FIG. 8B, electrode assembly 870 (e.g., including the exposed portion of electrically conductive portion 871) defines electrically conductive surface 875. Electrically conductive surface 875 can be configured to transmit and/or receive electrical signals, such as in examples in which electrode assembly 870 is positioned on expandable structure 190 of endovascular therapy system 100 (e.g., in which endovascular therapy system 100 is configured for endovascular stimulation therapy and/or sensing a patient parameter). As shown in the example of FIG. 8A and FIG. 8B, electrically conductive surface 875 is not covered by an electrically insulative material (e.g., not covered by electrically insulative portion 880 of electrode assembly 870). In some examples, electrically conductive surface 875 is configured to be placed proximate (e.g., in apposition with) a vessel wall, such as to maintain contact against a vessel wall for electrical stimulation therapy and/or sensing. When affixed to expandable structure 190, electrically conductive surface 875 is configured to face radially outward from expandable structure 190, such as in a direction radially outward from central longitudinal axis 191 of expandable structure 190 of FIG. 3. In the examples of FIG. 8A and FIG. 8B, electrically conductive surface 875 generally faces in the positive y-axis direction according to the orthogonal x-y-z axes shown in FIG. 8A and FIG. 8B.

In the example of FIG. 8A and FIG. 8B, electrically conductive surface 875 is a curved surface (e.g., which may correspond to a cylindrical-shape of electrically conductive portion 871 and/or circular or oval-shaped cross section of electrically conductive portion 871). However, in other examples, electrically conductive surface 875 is flat, concave, irregular, or defines another suitable shape.

Electrically conductive portion 871 of electrode assembly 870 includes a suitable electrically conductive material configured for transmitting and/or receiving electrical signals, such as those described with respect to electrically conductive portion 771 of FIG. 7A and FIG. 7B. In some examples, electrically conductive surface 875 includes a coating or other surface treatment to facilitate transmitting and/or receiving electrical signals, such as those described with respect to electrically conductive surface 775 of FIGS. 7A and 7B.

As shown in the examples of FIG. 8A and FIG. 8B, electrode assembly 870 includes electrically insulative portion 880. In some examples, electrically insulative portion 880 defines electrically insulative surface 876. In some examples, electrically insulative surface 876 is positioned opposite electrically conductive surface 875 (e.g., on an opposite side of electrode assembly 870 as electrically conductive surface 875). For example, electrically insulative portion 880 may cover a portion of electrically conductive portion 871, such as a surface of electrically conductive portion 871 facing in the negative y-axis direction according to the orthogonal x-y-z axes shown in FIG. 8A and FIG. 8B. Covering the surface of electrically conductive portion 871 facing in the negative y-axis direction with the electrically insulative material of electrically insulative portion 880 which may reduce or inhibit transmission of electrical signals to and/or from the surface of electrically conductive portion 871 facing in the negative y-axis direction.

When electrode assembly 870 is affixed to expandable structure 190, electrically insulative surface 876, which may be defined by electrically insulative portion 880, is configured to face radially inward from expandable structure 190, such as in a direction radially inward toward central longitudinal axis 191 of expandable structure 190. Electrically insulative portion 880 may reduce or even prevent transmission of electrical signals to and/or from less desirable regions of tissue and/or untargeted regions of tissue (e.g., a radially inward portion of a blood vessel or one or more nerves or brain structures closer to electrically insulative surface 876 than to electrically conductive portion 875). In some examples, electrically insulative portion 880 is applied to one or more sides of the of electrically conductive portion 871 such that electrically conductive surface 875 generally faces in a direction radially outward from expandable structure 190. In this way, electrode assembly 870 can bias transmissions of electrical signals to and/or from tissue surrounding the blood vessel (jugular vein 13) as compared to radially inward from the blood vessel wall. The biasing of transmissions of electrical signals to and/or from tissue surrounding the blood vessel can help facilitate directional electrical stimulation and/or sensing.

Electrically insulative portion 880 includes any suitable material or combination of materials configured for electrically insulating at least a portion of electrode assembly 870 from another structure (e.g., one or more portions of expandable structure 190 including projection 196, as well as other structures including conductor wires 166, anatomical features, biological tissue, etc.). Example materials for electrically insulative portion 880 include those described with respect to electrically insulative portion 780 of FIGS. 7A and 7B

Electrically insulative portion 880 is configured to be formed from any suitable processes or combination of processes. In some examples, electrically insulative portion 880 is formed from an injection molding process. In some examples, electrically insulative portion 880 of electrode assembly 870 includes an injection molded polymer. In some examples, electrically insulative portion 880 is configured to be injection molded (e.g., insert molded) around electrically conductive portion 871 (e.g., such that electrically conductive portion 871 can be placed into an injection molding mold, and electrically insulative portion 880 is formed around at least a portion of electrically conductive portion 871). Using a suitable injection molding process for forming electrically insulative portion 880 may enable electrode assembly 870 to define relatively complex geometries and/or relatively complex overall shape while allowing electrically conductive portion 871 to be a relatively simply geometry (e.g., a relatively simply geometrical shape such as a cylinder or disk). As creating complex geometries with injection molding of suitable polymers can be relatively easier than creating complex geometries of electrically conductive materials (e.g., PtIr or other electrically conductive materials discussed herein), the configurations of electrode assemblies discussed herein can facilitate relatively easier and/or simpler manufacturing and/or assembly of such electrode assemblies.

In other examples, electrically insulative portion 880 is configured to be formed (e.g., by a suitable forming process such as injection molding) and subsequently coupled with electrically conductive portion 871. For example, electrically insulative portion 880 can be formed via a suitable process and subsequently coupled with (e.g., via press fit, adhesive, or another suitable mechanical connection) with electrically conductive portion 871.

Electrode assembly 870 is configured to receive one or more structural features of a medical lead and/or other structures (e.g., features of medical lead 160 and/or expandable structure 190 of FIG. 3), such as for mechanically coupling one or more of electrode assembly 870 to medical lead 160 and/or expandable structure 190. The mechanical coupling of one or more of electrode assembly 870 to expandable structure 190 according to the techniques of this disclosure can help orient electrode assembly 870 with respect to the vasculature such that electrically conductive surface 875 faces radially outward and towards a vessel wall when expandable structure 190 with electrode assembly 870 are placed and deployed within the vasculature.

In some examples, electrode assembly 870 includes one or more structural features configured to receive portions of an expandable structure (e.g., expandable structure 190 of FIG. 3). In the example of FIG. 8A and FIG. 8B, electrode assembly 870 (e.g., electrically insulative portion 880) defines a fixation hole 882, which is configured to receive a respective portion of electrode attachment elements 194 of FIG. 3. For example, in some examples, fixation hole 882 is configured to receive projection 196 of electrode attachment element 194E.

Fixation hole 882 may be sized, oriented, and otherwise configured for receiving mating portions of expandable structure 190 (e.g., projection 196 of electrode attachment element 194E). In some examples, fixation hole 882 defines a maximum dimension L1 (which may be an internal maximum dimension). Maximum dimension L1 may be a diameter in examples in which fixation hole 882 defines a circular or approximately circular shape (e.g., a circular or approximately circular cross-section in a plane defined by the x and y axes according to the orthogonal x-y-z axes in the example of FIG. 8A and FIB. 8B).

In some examples, electrically insulative portion 880, which may be configured to receive a portion of an electrode attachment element 194E including projection 196, is configured to deform to create an interference fit between electrically insulative portion 880 and a portion of electrode attachment element 194E, such as projection 196. As discussed in relation to some previous examples (e.g., FIG. 4A and FIG. 4B), a projection (e.g., projection 496) can include features (e.g., barbs 497) that facilitate the interference fit between the projection and electrically insulative portion 880. In some examples, as discussed above with respect to FIG. FIG. 4A and FIG. 4B, projection 496 (including barbs 497) can be configured to mate with electrically insulative portion 880 (e.g., one or more internal notches or pockets defined by electrically insulative portion 880, such as to create a snap fit). In some examples, maximum dimension L1 changes upon insertion of a respective mating feature (e.g., projection 196 of electrode attachment element 194E).

In some examples, maximum dimension L1 corresponds to a maximum outer dimension of a respective mating feature (e.g., projection 196 of electrode attachment element 194E). In some examples, maximum dimension L1 is equal to or slightly larger than the respective mating feature (e.g., projection 196). In other examples, maximum dimension L1 is equal to or slightly smaller than the respective mating feature (e.g., projection 196), such as in examples in which fixation hole 882 is configured to create an interference fit with the respective mating feature (e.g., projection 196).

In some examples, as shown in the example of FIG. 8A and FIG. 8B, fixation hole 882 is a through hole and extends entirely through electrode assembly 870 (e.g., extends entirely through electrically insulative portion 880 in a direction along the z-axis according to the orthogonal x-y-z axes shown in FIG. 8A and FIG. 8B). In other examples, fixation hole 882 extends only partially through electrode assembly 870 (e.g., only partially through electrically insulative portion 882 in a direction along the z-axis), such that fixation hole 882 is a blind hole. In some examples, using blind holes can minimize and/or prevent fluid ingress such as to minimize and/or prevent interference with the mechanical connection between electrode assemblies 870 and expandable structure 190, as well as to help prevent thrombus formation.

In some examples, at least a portion of electrode assembly 870 (e.g., electrically insulative portion 880) is configured to interface with (e.g., rest on, contact, receive, and/or otherwise engage with) a strut (e.g., one of struts 192) of an expandable structure (e.g., expandable structure 190). For example, when electrode assembly 870 is attached to expandable structure 190, at least a portion of electrically insulative portion 880 rests on, contacts, receives, and/or otherwise engages with one of struts 192 of expandable structure 190. In some examples, at least a portion of electrically insulative portion 880 defines a shape that conforms to (e.g., wraps around) at least a portion of one of struts 192. By engaging both an electrode attachment element (e.g., projection 196 via fixation hole 882) and one of struts 192, electrically insulative portion 880 is configured to minimize rotation of electrode assembly 870 relative to expandable structure 190. In this way, electrode assembly 870 is fixed via at least two connection points to expandable structure 190, which may minimize, or even prevent, rotation of electrode assembly 870 with respect to one of struts 192. By being fixed at two spaced-apart points, electrode assembly 870 may exhibit minimal or no rotation or other movement with respect to one of struts 192 and/or electrode attachment elements 194 of expandable structure 190 (e.g., during deployment of expandable structure 190 or while experiencing external forces, such as from a vessel wall when one or more of electrode assembly 870 is brought into contract with the vessel wall).

In some examples, electrically insulative portion 880 defines one or more slots 887 (shown as first slot 887A and/or second slot 887B in the example of FIG. 8A and FIG. 8B, but collectively referred to herein as one or more slots 887). Slots 887 can be configured to engage with (e.g., wrap around) at least a portion of an expandable structure, including one of struts 192 of expandable structure 190, such as to prevent rotation of electrode assembly 870 with respective to one of struts 192. In some examples, slots 887 are configured to receive a portion of one of struts 192. For example, in examples in which electrode assembly 870 is mechanically coupled to electrode attachment element 194E, and fixation hole 882 has received projection 196, either of first slot 887A or second slot 887B (e.g., whichever is positioned closest to the corresponding strut 192A) is configured to receive a portion of strut 192A.

As shown in the example of FIG. 3, electrode assembly 170B and electrode assembly 170D may be examples of electrode assembly 870, where electrode assembly 870 is configured to engage both with projection 196 of one of electrode attachment elements 194 and one of strut 192 of expandable structure 190. In this way, each of first slot 887A or second slot 887B are configured to minimize rotation of electrode assembly 870 around one of strut 192.

In some cases, engagement of one slot 887A or 887B with a strut may be sufficient to help prevent rotation of electrode assembly 870 relative to expandable structure 190. Thus, in some examples, when loaded onto electrode attachment element 194E, one of first slot 887A or second slot 887B may not be in use (e.g., may not engage one of struts 192). As explained more fully below, each of first slot 887A or second slot 887B can be symmetrical with respect to each other, as well as symmetrical with respect to electrode assembly 870 as a whole (as shown in FIG. 8A and FIG. 8B) which may enable electrode assembly 870 to be coupled to electrode attachment element 194E in at least two different orientations (e.g., at least a first orientation and a second orientation). For example, in the first orientation (e.g., a first orientation in which a first face of insulative portion 880 facing in the positive z-axis direction as the leading face when electrode assembly 870 is loaded first onto projection 196), only one of slots 887 (e.g., first slot 887A) engages with one of struts 192, while the other slot (e.g., second slot 887B) does not engage one of struts 192. In a second orientation (e.g., a second orientation in which a second face of insulative portion 880 facing in the negative z-axis direction as the leading face when electrode assembly 870 is loaded first onto projection 196), only one of slots 887 (e.g., second slot 887B) engages with one of struts 192, while the other slot (e.g., first slot 887A) does not engage one of struts 192.

Slots 887 can be defined by electrode assembly 870 using any suitable structure. As shown in the example of FIG. 8A and FIG. 8B, insulative portion 880 of electrode assembly 870 can include wings 886 (shown individually as wing 886A, wing 886B, wing 886C, and wing 886D, but collectively referred to herein as wings 886). In some examples, pairs of wings 886 define each of slot 887A and slot 887B. For example, wing 886A and wing 886C extend in the negative x-axis direction away from a central longitudinal axis parallel to the z-axis, and extend along a length in the positive and negative z-axis directions according to the orthogonal x-y-x axes shown in FIGS. 8A and 8B to define slot 887A. Similarly, wing 886B and wing 886D extend in the positive x-axis direction away from the central longitudinal axis parallel to the z-axis, and extend along a length in the positive and negative z-axis directions according to the x-y-x axes shown in FIGS. 8A and 8B to define slot 887B. Wings 886 can be configured to rest on, contact, and/or otherwise engage with one or more of struts 192 of expandable structure 190. A pair of wings 886 (e.g., the pair of wing 886A and wing 886C or the pair of wing 886B and wing 886D) may be configured to sandwich one of struts 192. Wings 886 may have a suitable shape and size (e.g., thickness) to prevent rotation of electrode assembly 870 relative to one of struts 192 when either of slot 887A or slot 887B defined by wings 886 receives one of struts 192 of expandable structure 190.

In some examples, electrode assembly 870 is configured to electrically connect to one or more conductor wires, such as to facilitate electrical communication of electrode assembly 870 (e.g., electrically conductive portion 871) with a medical device (e.g., medical device 14 of FIG. 1). In some examples, a portion of electrode assembly 870 (e.g., a portion of one or more of electrically conductive portion 871 and electrically insulative portion 880) defines a conductor hole 874 (which may be referred to as a first conductor hole 874), where conductor hole 874 is configured to receive a conductor wire (e.g., one or more of conductor wires 166 of FIG. 3) for electrically coupling electrode assembly 870 to a medical device. One or more of conductor wires 166 can be mechanically coupled to electrode assembly 870 (e.g., once one of conductor wires is positioned in conductor hole 874) via a suitable mechanical connection (e.g., welding, adhesive, interference fit in conductor hole 874, or the like). In some examples, conductor hole 874 includes an electrical contact electrically connected to electrically conductive portion 871 of electrode assembly 870. Thus, when a conductor wire 166 is positioned in conductor hole 874 and electrically connected to the electrical contact, the conductor wire 166 is electrically connected to electrically conductive portion 871.

In some examples, conductor hole 874 is a through hole and extends entirely through electrode assembly 870 (e.g., extending entirely through electrode assembly 870 in a direction along the z-axis). In other examples, conductor hole 874 extends only partially through electrode assembly 870 (e.g., only partially through electrode assembly 870 in a direction along the z-axis), such that conductor hole 874 is a blind hole. In some examples, using blind holes can minimize and/or prevent fluid ingress such as to minimize and/or prevent interference with the electrical connection between electrode assemblies 870 and conductor wires 166 and help prevent thrombus formation. In some examples, conductor hole 874 is formed from both electrically conductive portion 871 and electrically insulative portion 880 of electrode assembly 870. In other examples, conductor hole 874 is formed from only one of electrically conductive portion 871 or electrically insulative portion 880.

In some examples, conductor hole 874 is sized to accommodate one conductor wire (e.g., of conductor wires 166). In some examples, conductor hole 874 defines a maximum dimension L2 (which may be an internal dimension of conductor hole 874). In some examples, maximum dimension L2 is slightly larger than a maximum cross-sectional dimension (e.g., a width) of one conductor wire (e.g., of conductor wires 166). In examples in which conductor hole 874 defines a circular or approximately circular shape, maximum dimension L2 is a diameter.

In some examples, conductor hole 874 is configured as a pass-through hole for one or more conductor wires (e.g., conductor wires 166 of FIG. 3). Conductor hole 874 being configured as a pass-through hole can facilitate electrical connection of the conductor wire (e.g., one or more of conductor wires 166) to a second, different electrode assembly. For example, one or more of conductor wires 166 may be fed through conductor hole 874 and electrically connected to a different (e.g., more distal) electrode. In this way, multiple of electrode assemblies can be electrically connected to a common conductor wire of conductor wires 166 (e.g., “shorted together”), such that more than electrode assemblies can be controlled together (e.g., by a medical device, such as medical device 14 of FIG. 1).

In some examples, electrode assembly 870 defines at least a second conductor hole 884, where second conductor hole 884 is configured as a pass-through hole for one or more conductor wires (e.g., conductor wires 166 of FIG. 3). Second conductor hole 884 may facilitate routing of conductor wires to at least a second, different electrode assembly (e.g., in examples in which electrode assemblies are not “shorted” together and are controlled separately by medical device 14). For example, as shown in the example of FIG. 3, conductor wire 166C, which is associated with and electrically connected to electrode assembly 170C, can be routed through a pass-through conductor hole of a more proximal electrode assembly (e.g., electrode assembly 170D, of which electrode assembly 870 may be an example). Second conductor hole 884 being configured as a pass-through hole can reduce crossing or tangling of conductor wires (e.g., conductor wires 166) with each other and/or with portions of expandable structure 190, and may otherwise facilitate management of conductor wires (e.g., conductor wires 166) proximate an expandable structure (e.g., expandable structure 190).

In some examples, electrode assembly 870 is sized, shaped, or otherwise configured to facilitate relatively faster and/or relatively easier fabrication of one or more of electrode assembly 870 and/or assembly with other portions of a medical device system (e.g., expandable structure 190). In some examples, electrode assembly 870 is sized, shaped, or otherwise configured to facilitate relatively easy attachment to expandable structure 190 (e.g., such as to reduce assembly errors, poka-yoke, or the like). For example, as explained below, electrode assembly can include symmetry and/or redundant features that enable electrode assembly 870 to be mechanically coupled to expandable structure 190 in more than one orientation, and electrode assembly 870 may function similarly or the same in each of the one or more orientations.

In some examples, at least a portion of electrode assembly 870 is symmetric (e.g., reflectionally symmetric). As shown in FIG. 8B, electrode assembly 870 is symmetric (e.g., reflectionally symmetric) about (e.g., relative to) a medial plane 879 intersecting a midpoint on an electrode assembly face (e.g., the face of electrode assembly 870 facing in the positive z-axis direction). In the example of FIG. 8B, medial plane 879 is parallel to the y-axis and extends through electrode assembly 870 in the z-axis direction. In some examples, as shown in the example of FIG. 8A and FIG. 8B, electrode assembly 870 is symmetric such that first slot 887A and second slot 887B are equidistant from a medial center of electrode assembly 870 (e.g., respective centers of each of first slot 887A and second slot 887B in a plane defined by the x-axis and the y-axis are equidistance from medial plane 879). Additionally, slots 887 are equidistant from, and symmetric with respect to, each of first conductor hole 874 and second conductor hole 884. Said another way, each of first conductor hole 874 and second conductor hole 884 are positioned between and equidistant from each of slots 887. Reflectional symmetry as described herein may enable electrode assembly 870 to be attached to expandable structure 190 in at least two different orientations. For example, electrode assembly 870 can be attached to projection 196 of electrode attachment element 194E with either the face of electrode assembly 870 (e.g., either face of insulative portion 880) in the positive z-axis direction or the negative z-axis direction as the leading face. Symmetry of electrode assembly 870 may also facilitate balance of electrode assembly 870 when fixed to one of electrode attachment element 194.

In some examples, as shown in FIG. 8B, each of first conductor hole 874 and second conductor hole 884 (which may be a pass-through conductor hole) are symmetric (e.g., reflectionally symmetric) about medial plane 879. In some examples, one or more of first conductor hole 874 and/or second conductor hole 884 intersects medial plane 879. While the example of FIG. 8A and FIG. 8B depict electrode assembly 870 as symmetric about medial plane 879, other configurations (including other symmetrical configurations) are contemplated.

In other examples, electrode assembly 870, including one or more structural features of electrode assembly 870, is asymmetric (e.g., asymmetric about medial plane 879). For example, electrode assembly 870 can be asymmetric such that first slot 887A and second slot 887B are not equidistant from a medial center of electrode assembly 870 (e.g., not equidistance from medial plane 879). In some examples, conductor hole 874 is not symmetric about medial plane 879 and/or does not intersect medial plane 879. In some examples, electrically conductive surface 875 is asymmetric relative to medial plane 879 (e.g., electrically conductive surface 875 extends relatively more in the positive x-axis direction or the negative x-axis direction in the example of FIG. 8A and FIG. 8B). In some examples, asymmetry of electrode assembly 870 (including electrically conductive surface 875) facilitates a relative larger conductive surface (e.g., electrically conductive surface 875) as compared to other configurations.

FIG. 9A and FIG. 9B illustrate a perspective view and a side view, respectively, of an electrode assembly 970, which is another example of one of electrode assemblies 17 of FIG. 1 and/or one of electrode assemblies 170 of FIG. 3. Electrode assembly 970 includes an electrically conductive portion 971 that defines an electrically conductive surface 975 (which may be referred to as a first surface). Electrode assembly 970 includes an electrically insulative portion 980 configured to electrically insulate at least a portion of electrically conductive portion 971 from another structure (e.g., a portion of lead 160, expandable structure 190 of FIG. 3, an anatomical structure, biological matter, and/or the like). Electrically insulative portion 980 defines at least one electrically insulative surface 976 (which may be referred to as a second surface), which may be positioned opposite electrically conductive surface 975, as described more fully herein.

For illustrative purposes, some elements described in FIG. 3 will be referenced herein with respect to electrode assembly 970 (e.g., as corresponding mating structural features), however it should be understood that electrode assembly 970 is only one example of electrode assemblies 170 of FIG. 3, and that other types of electrode assemblies can additionally or alternatively be used in conjunction with the elements described in connection with FIG. 3. Additionally, some elements of FIG. 6 will be referenced herein to illustrate how electrode assembly 970 can be configured to receive multiple projections of an electrode attachment element (e.g., electrode assembly 970 can be configured to receive each of first projection 696A, second projection 696B, and third projection 696C of electrode attachment element 694 of FIG. 6).

Electrode assembly 970 is configured to mechanically couple to another structure (e.g., a medical lead or expandable structure, such as medical lead 160 and/or expandable structure 190 of FIG. 3). In some examples, electrode assembly 970 includes one or more structural features configured to facilitate mechanical coupling of electrode assembly 970 to another structure (e.g., a medical lead or expandable structure, such as medical lead 160 and/or expandable structure 190 of FIG. 3). For example, electrically insulative portion 980 of electrode assembly 970 is configured to facilitate mechanical coupling of electrode assembly 970 to expandable structure 190. Electrically insulative portion 980 can also be configured to electrically insulate one or more electrically conductive portions of electrode assembly 970 (e.g., electrically conductive portion 971) from portions of expandable structure 190. In this way, electrically insulative portion 980 can be configured to both facilitate mechanical coupling of electrode assembly 970 to expandable structure 190 as well facilitate electrical insulation of at least a portion of electrode assembly 970 from another structure (e.g., at least a portion of expandable structure 190). This dual purpose can reduce or even eliminate the need to provide separate features and/or separate components to serve each of these mechanical coupling and electrically insulating functions. This dual purpose may shorten the time needed for manufacturing and/or simplify manufacturing and/or assembly of electrode assembly 970. Further, this dual purpose served by electrically insulative portion 980 can also help maintain a relatively low profile electrode assembly 970 and endovascular device including electrode assembly 970. A relatively low profile electrode assembly 970 can be particularly advantageous when used in vasculature of a patient. For example, the low profile can facilitate easier navigation to a target site in vasculature of a patient as well as minimize adverse impacts to blood flow in the vasculature by the endovascular device that includes the relatively low profile electrode assembly 970.

Prior to assembly into the final form factor of electrode assembly 970, electrically conductive portion 971 can define any suitable shape. In some examples, electrically conductive portion 971 defines a tubular (e.g., cylindrical) shape, a half-cylinder shape, ring-like shape, or the like. In some examples, electrically conductive portion 971 includes a hollow cylinder. In some examples, as discussed herein, at least a sub-portion electrically conductive portion 971 is configured to be covered by an electrically insulative material (e.g., electrically insulative material 980). In some examples, the electrically insulative material (e.g., electrically insulative material 980) covers at least a portion of electrically conductive portion 971. For example, as shown in the example FIG. 9A and FIG. 9B, electrically insulative portion 980 covers at least an interior portion (e.g., radially inward portion) as well as an exterior portion (e.g., radially outward portion) of electrically conductive portion 971. In examples in which electrically conductive portion 971 defines a hollow cylinder shape, electrically insulative portion 980 covers at least a portion of the interior and the exterior of the hollow cylinder shape.

Electrically conductive portion 971 (which can include a cylindrical shape) can be formed using any suitable technique. In some examples, the electrically conductive portion 971 is formed via a machining or stamping process.

As shown in the example of FIG. 9A and FIG. 9B, electrode assembly 970 (e.g., including the exposed portion of electrically conductive portion 971) defines electrically conductive surface 975. Electrically conductive surface 975 can be configured to transmit and/or receive electrical signals, such as examples in which electrode assembly 970 is positioned on expandable structure 190 of endovascular therapy system 100 (e.g., in which endovascular therapy system 100 is configured for endovascular stimulation therapy and/or sensing a patient parameter). As shown in the example of FIG. 9A and FIG. 9B, electrically conductive surface 975 is not covered by an electrically insulative material (e.g., not covered by electrically insulative portion 980 of electrode assembly 970). In some examples, electrically conductive surface 975 is configured to be placed proximate (e.g., in apposition with) a vessel wall, such as to maintain contact against a vessel wall for electrical stimulation therapy and/or sensing. When affixed to expandable structure 190, electrically conductive surface 975 is configured to face radially outward from expandable structure 190, such as in a direction radially outward from central longitudinal axis 191 of expandable structure 190 of FIG. 3. In the examples of FIG. 9A and FIG. 9B, electrically conductive surface 975 generally faces in the positive y-axis direction according to the orthogonal x-y-z axes shown in FIG. 9A and FIG. 9B.

In the example of FIG. 9A and FIG. 9B, electrically conductive surface 975 is a curved surface (e.g., which may correspond to a cylindrical-shape of electrically conductive portion 971 and/or circular or oval-shaped cross section of electrically conductive portion 971). However, in other examples, electrically conductive surface 975 is flat, concave, irregular, or defines another suitable shape.

Electrically conductive portion 971 of electrode assembly 970 includes a suitable electrically conductive material configured for transmitting and/or receiving electrical signals, such as those described with respect to electrically conductive portion 771 of FIG. 7A and FIG. 7B. In some examples, electrically conductive surface 975 includes a coating or other surface treatment to facilitate transmitting and/or receiving electrical signals, such as those described with respect to electrically conductive surface 775 of FIGS. 7A and 7B.

As shown in the examples of FIG. 9A and FIG. 9B, electrode assembly 970 includes electrically insulative portion 980. In some examples, electrically insulative portion 980 defines electrically insulative surface 976. In some examples, electrically insulative surface 976 is positioned opposite electrically conductive surface 975 (e.g., on an opposite side of electrode assembly 970 as electrically conductive surface 975). For example, electrically insulative portion 980 may cover a portion of electrically conductive portion 971, such as a surface of electrically conductive portion 971 facing in the negative y-axis direction according to the orthogonal x-y-z axes shown in FIG. 9A and FIG. 9B. Covering the surface of electrically conductive portion 971 facing in the negative y-axis direction with the electrically insulative material of electrically insulative portion 980 which may reduce or inhibit transmission of electrical signals to and/or from the surface of electrically conductive portion 971 facing in the negative y-axis direction.

When electrode assembly 970 is affixed to expandable structure 190, electrically insulative surface 976, which may be defined by electrically insulative portion 980, is configured to face radially inward from expandable structure 190, such as in a direction radially inward toward central longitudinal axis 191 of expandable structure 190. Electrically insulative portion 980 may reduce or even prevent transmission of electrical signals to and/or from less desirable regions of tissue and/or untargeted regions of tissue (e.g., a radially inward portion of a blood vessel or one or more nerves or brain structures closer to electrically insulative surface 976 than to electrically conductive portion 975). In some examples, electrically insulative portion 980 is applied to one or more sides of the of electrically conductive portion 971 such that electrically conductive surface 975 generally faces in a direction radially outward from expandable structure 190. In this way, electrode assembly 970 can bias transmissions of electrical signals to and/or from tissue surrounding the blood vessel (jugular vein 13) as compared to radially inward from the blood vessel wall. The biasing of transmissions of electrical signals to and/or from tissue surrounding the blood vessel can help facilitate directional electrical stimulation and/or sensing.

Electrically insulative portion 980 includes any suitable material or combination of materials configured for electrically insulating at least a portion of electrode assembly 970 from another structure (e.g., one or more portions of expandable structure 190 of FIG. 3, one or more projections of an electrode attachment element, such as one or more of first projection 696A, second projection 696B, or third projection 696C of electrode attachment element 694 of FIG. 6, as well as other structures including conductor wires 166 of FIG. 3, anatomical features, biological tissue, etc.). Example materials for electrically insulative portion 980 include those described with respect to electrically insulative portion 780 of FIGS. 7A and 7B.

Electrically insulative portion 980 is configured to be formed from any suitable processes or combination of processes. In some examples, electrically insulative portion 980 is formed from an injection molding process. In some examples, electrically insulative portion 980 of electrode assembly 970 includes an injection molded polymer. In some examples, electrically insulative portion 980 is configured to be injection molded (e.g., insert molded) around electrically conductive portion 971 (e.g., such that electrically conductive portion 971 can be placed into an injection molding mold, and electrically insulative portion 980 is formed around at least a portion of electrically conductive portion 971). Using a suitable injection molding process for forming electrically insulative portion 980 may enable electrode assembly 970 to define relatively complex geometries and/or relatively complex overall shape while allowing electrically conductive portion 971 to be a relatively simply geometry (e.g., a relatively simply geometrical shape such as a cylinder or disk). As creating complex geometries with injection molding of suitable polymers can be relatively easier than creating complex geometries of electrically conductive materials (e.g., PtIr or other electrically conductive materials discussed herein), the configurations of electrode assemblies discussed herein can facilitate relatively easier and/or simpler manufacturing and/or assembly of such electrode assemblies.

In other examples, electrically insulative portion 980 is configured to be formed (e.g., by a suitable forming process such as injection molding) and subsequently mechanically coupled with electrically conductive portion 971. For example, electrically insulative portion 980 can be formed via a suitable process and subsequently coupled with (e.g., via press fit, adhesive, or another suitable mechanical connection) with electrically conductive portion 971.

Electrode assembly 970 is configured to receive one or more structural features of a medical lead and/or other structures (e.g., features of medical lead 160 and/or expandable structure 190 of FIG. 3), such as for mechanically coupling one or more of electrode assembly 970 to medical lead 160 and/or expandable structure 190. The mechanical coupling of one or more of electrode assembly 970 to expandable structure 190 according to the techniques of this disclosure can help orient electrode assembly 970 with respect to the vasculature such that electrically conductive surface 975 faces radially outward and towards a vessel wall when expandable structure 190 with electrode assembly 970 are placed and deployed within the vasculature.

In some examples, electrode assembly 970 includes one or more structural features configured to receive portions of an expandable structure (e.g., expandable structure 190 of FIG. 3). In the example of FIG. 9A and FIG. 9B, electrode assembly 970 (e.g., electrically insulative portion 980) defines a first fixation hole 982A, a second fixation hole 982B, and a third fixation hole 982C (collectively referred to herein as fixation holes 982). Each of first fixation hole 982A, second fixation hole 982B, and third fixation hole 982C may be configured to receive respective portions of an electrode attachment element (e.g., first projection 696A, second projection 696B, and third projection 696C of electrode attachment element 694 of FIG. 6). For example, in some examples, first fixation hole 982A is configured to receive first projection 696A of electrode attachment element 694, second fixation hole 982B is configured to receive second projection 696B of electrode attachment element 694, and third fixation hole 982C is configured to receive third projection 696C of electrode attachment element 694.

Fixation holes 982 may be sized, oriented, and otherwise configured for receiving mating portions of expandable structure 190 (e.g., first projection 696A, second projection 696B, and third projection 696C of electrode attachment element 694 of FIG. 6). In some examples, one or more of fixation holes 982 defines a maximum dimension L1 (which may be an internal maximum dimension). Maximum dimension L1 may be a diameter in examples in which one or more fixation holes 982 defines a circular or approximately circular shape (e.g., a circular or approximately circular cross-section in a plane defined by the x and y axes according to the orthogonal x-y-z axes in the example of FIG. 9A and FIB. 9B).

In some examples, electrically insulative portion 980, which may be configured to receive a portion of an electrode attachment element (e.g., electrode attachment element 694 including one or more of first projection 696A, second projection 696B, and/or third projection 696C as shown in FIG. 6), is configured to deform to create an interference fit between electrically insulative portion 980 and a portion of electrode attachment element 694. As discussed in relation to some previous examples (e.g., FIG. 4A and FIG. 4B), a projection (e.g., one or more of one or more of first projection 696A, second projection 696B, and/or third projection 696C as shown in FIG. 6) can include features (e.g., barbs 497 as shown in FIG. 4A and FIG. 4B) that facilitate the interference fit between the one or more of projections 696 and electrically insulative portion 980. In some examples, as discussed above with respect to FIG. FIG. 4A and FIG. 4B, projection 496 (including barbs 497) can be configured to mate with electrically insulative portion 980 (e.g., one or more internal notches or pockets defined by electrically insulative portion 980, such as to create a snap fit). In some examples, maximum dimension L1 changes upon insertion of a respective mating feature (e.g., one or more of projections 696 of electrode attachment element 694).

In some examples, maximum dimension L1 corresponds to a maximum outer dimension of a respective mating feature (e.g., one or more of projections 696 of electrode attachment element 694). In some examples, maximum dimension L1 is equal to or slightly larger than the respective mating feature (e.g., one or more of projections 696 of electrode attachment element 694). In other examples, maximum dimension L1 is equal to or slightly smaller than the respective mating feature (e.g., one or more of projections 696 of electrode attachment element 694), such as in examples in which fixation hole 982 is configured to create an interference fit with the respective mating feature (e.g., one or more of projections 696 of electrode attachment element 694).

In some examples, as shown in the example of FIG. 9A and FIG. 9B, each of fixation holes 982 are through holes and extend entirely through electrode assembly 970 (e.g., extend entirely through electrically insulative portion 980 in a direction along the z-axis). In other examples, one or more of fixation holes 982 extend only partially through electrode assembly 970 (e.g., only partially through electrically insulative portion 980 in a direction along the z-axis), such that one or more of fixation holes 982 is a blind hole. In some examples, using blind holes can minimize and/or prevent fluid ingress such as to minimize and/or prevent interference with the mechanical connection between electrode assemblies 970 and expandable structure 190, and/or to help prevent thrombus formation.

In the example of FIG. 9A and FIG. 9B, electrode assembly 970 is configured to be fixed at two or more points relative to an expandable structure (e.g., via multiple projections of an electrode attachment element, such as via projections 696 of electrode attachment element 694 of FIG. 6). In this way, electrode assembly 970 can be fixed via at least two connection points to expandable structure 190, which may minimize, or even prevent, rotation of electrode assembly 970 with respect to at least a portion of expandable structure 190 (e.g., such as strut 692 of FIG. 6). By being fixed at two spaced-apart points, electrode assembly 970 may exhibit minimal or no rotation or other movement with respect to at least a portion of expandable structure 190 (e.g., during deployment of expandable structure 190 or while experiencing external forces, such as from a vessel wall when one or more of electrode assembly 970 is brought into contract with the vessel wall).

As shown in the example of FIG. 9A and FIG. 9B, insulative portion 980 of electrode assembly 970 can include wings 986 (shown individually as first wing 986A and second wing 986B but collectively referred to herein as wings 986). In some examples, first wing 986A defines second fixation hole 982B and second wing 986B defines third fixation hole 982C.

In some examples, electrode assembly 970 is configured to electrically connect to one or more conductor wires, such as to facilitate electrical communication of electrode assembly 970 (e.g., electrically conductive portion 971) with a medical device (e.g., medical device 14 of FIG. 1). In some examples, a portion of electrode assembly 970 (e.g., a portion of one or more of electrically conductive portion 971 and electrically insulative portion 980) defines a conductor hole 974 (which may be referred to as a first conductor hole 974), where conductor hole 974 is configured to receive a conductor wire (e.g., one or more of conductor wires 166 of FIG. 3) for electrically coupling electrode assembly 970 to a medical device. One or more of conductor wires 166 can be mechanically coupled to electrode assembly 970 (e.g., once one of conductor wires is positioned in conductor hole 974) via a suitable mechanical connection (e.g., welding, adhesive, interference fit in conductor hole 974, or the like). In some examples, conductor hole 974 includes an electrical contact electrically connected to electrically conductive portion 971 of electrode assembly 970. Thus, when a conductor wire 166 is positioned in conductor hole 974 and electrically connected to the electrical contact, the conductor wire 166 is electrically connected to electrically conductive portion 971.

In some examples, conductor hole 974 is a through hole and extends entirely through electrode assembly 970 (e.g., extending entirely through electrode assembly 970 in a direction along the z-axis). In other examples, conductor hole 974 extends only partially through electrode assembly 970 (e.g., only partially through electrode assembly 970 in a direction along the z-axis), such that conductor hole 974 is a blind hole. In some examples, using blind holes can minimize and/or prevent fluid ingress such as to minimize and/or prevent interference with the electrical connection between electrode assemblies 970 and conductor wires 166 and help prevent thrombus formation. In some examples, conductor hole 974 is formed from both electrically conductive portion 971 and electrically insulative portion 980 of electrode assembly 970. In other examples, conductor hole 974 is formed from only one of electrically conductive portion 971 or electrically insulative portion 980.

In some examples, conductor hole 974 is sized to accommodate one conductor wire (e.g., of conductor wires 166). In some examples, conductor hole 974 defines a maximum dimension L2 (which may be an internal dimension of conductor hole 974). In some examples, maximum dimension L2 is slightly larger than a maximum cross-sectional dimension (e.g., a width) of one conductor wire (e.g., of conductor wires 166). In examples in which conductor hole 974 defines a circular or approximately circular shape, maximum dimension L2 is a diameter.

In some examples, conductor hole 974 is configured as a pass-through hole for one or more conductor wires (e.g., conductor wires 166 of FIG. 3). Conductor hole 974 being configured as a pass-through hole can facilitate electrical connection of the conductor wire (e.g., one or more of conductor wires 166) to a second, different electrode assembly. For example, one or more of conductor wires 166 may be fed through conductor hole 974 and electrically connected to a different (e.g., more distal) electrode. In this way, multiple of electrode assemblies can be electrically connected to a common conductor wire of conductor wires 166 (e.g., “shorted together”), such that more than electrode assemblies can be controlled together (e.g., by a medical device, such as medical device 14 of FIG. 1).

In some examples, electrode assembly 970 defines at least a second conductor hole 984, where second conductor hole 984 is configured as a pass-through hole for one or more conductor wires (e.g., conductor wires 166 of FIG. 3). Second conductor hole 984 may facilitate routing of conductor wires to at least a second, different electrode assembly (e.g., in examples in which electrode assemblies are not “shorted” together and are controlled separately by medical device 14). For example, as shown in the example of FIG. 3, conductor wire 166C, which is associated with and electrically connected to electrode assembly 170C, can be routed through a pass-through conductor hole of a more proximal electrode assembly (e.g., electrode assembly 170D, of which electrode assembly 970 may be an example). Second conductor hole 984 being configured as a pass-through hole can reduce crossing or tangling of conductor wires (e.g., conductor wires 166) with each other and/or with portions of expandable structure 190, and may otherwise facilitate management of conductor wires (e.g., conductor wires 166) proximate an expandable structure (e.g., expandable structure 190).

In some examples, electrode assembly 970 is sized, shaped, or otherwise configured to facilitate relatively faster and/or relatively easier fabrication of one or more of electrode assembly 970 and/or assembly with other portions of a medical device system (e.g., expandable structure 190). In some examples, electrode assembly 970 is sized, shaped, or otherwise configured to facilitate relatively easy attachment to expandable structure 190 (e.g., such as to reduce assembly errors, poka-yoke, or the like). For example, as explained below, electrode assembly can include symmetry and/or redundant features that enable electrode assembly 970 to be mechanically coupled to expandable structure 190 in more than one orientation, and electrode assembly 970 may function similarly or the same in each of the one or more orientations.

In some examples, at least a portion of electrode assembly 970 is symmetric (e.g., reflectionally symmetric). As shown in FIG. 9B, electrode assembly 970 is symmetric (e.g., reflectionally symmetric) about (e.g., relative to) a medial plane 979 intersecting a midpoint on an electrode assembly face (e.g., the face of electrode assembly 970 facing in the positive z-axis). In the example of FIG. 9B, medial plane 979 is parallel to the y-axis and extends through electrode assembly 970 in the z-axis direction. In some examples, as shown, electrode assembly 970 is symmetric such that second fixation hole 982B and third fixation hole 982C are equidistant from a medial center of electrode assembly 970 (e.g., respective centers of each of second fixation hole 982B and third fixation hole 982C in a plane defined by the x-axis and the y-axis are equidistance from medial plane 979). This reflectional symmetry may enable electrode assembly 970 to be attached to expandable structure 190 in at least two different orientations. For example, electrode assembly 970 can be attached to projections 696 of electrode attachment element 694 of FIG. 6 with either the face of electrode assembly 970 (e.g., either face insulative portion 980) in the positive z-axis direction or the negative z-axis direction as the leading face.

In some examples, as shown in FIG. 9B, each of first conductor hole 974 and second conductor hole 984 (which may be a pass-through conductor hole) are symmetric (e.g., reflectionally symmetric) about medial plane 979. In some examples, one or more of first conductor hole 974 and/or second conductor hole 984 intersects medial plane 979. While the example of FIG. 9A and FIG. 9B depict electrode assembly 970 as symmetric about medial plane 979, other configurations (including other symmetrical configurations) are contemplated.

In other examples, electrode assembly 970, including one or more structural features of electrode assembly 970, is asymmetric (e.g., asymmetric about medial plane 979). For example, electrode assembly 970 can be asymmetric such that second fixation hole 982B and third fixation hole 982C are not equidistant from a medial center of electrode assembly 970 (e.g., not equidistance from medial plane 979). In some examples, conductor hole 974 is not symmetric about medial plane 979 and/or does not intersect medial plane 979. In some examples, electrically conductive surface 975 is asymmetric relative to medial plane 979 (e.g., electrically conductive surface 975 extends relatively more in the positive x-axis direction or the negative x-axis direction in the example of FIG. 9A and FIG. 9B). In some examples, asymmetry of electrode assembly 970 (including electrically conductive surface 975) facilitates a relative larger conductive surface (e.g., electrically conductive surface 975) as compared to other configurations.

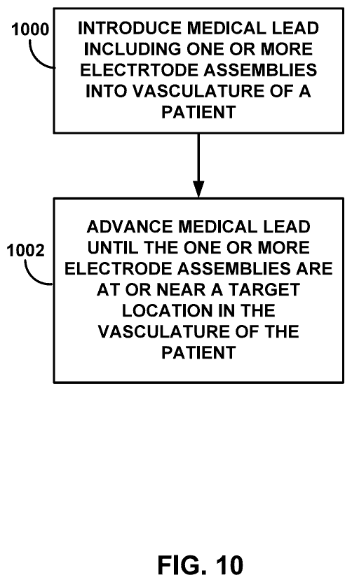

FIG. 10 is a flow diagram illustrating an example technique for using a medical device system according to the techniques of this disclosure, which may include placing a medical lead adjacent a target location in vasculature of a patient. The technique of FIG. 10 is described with respect to therapy system 10 of FIG. 1, as well as endovascular therapy system 100 of FIG. 3 (which is an example of therapy system 10 of FIG. 1), but may be used with any of the device, systems, and/or elements of systems described in this disclosure.

In the example of FIG. 10, the technique includes introducing an endovascular device (e.g., endovascular device 16 and/or medical lead 160) into vasculature of patient 12 (1000). For example, a clinician may introduce at least distal portion 150 medical lead 160 through an access point in patient 12 including a femoral artery access point or radial artery access point. In some examples, one or more of an introducer sheath, a guide catheter, and/or a guidewire is used to facilitate introduction of medical lead 160 into patient 12.

In the example of FIG. 10, the technique further includes advancing medical lead 160 through the vasculature of the patient until electrode assemblies 170 are adjacent a target location in the vasculature of patient 12 (1002). In some examples, a clinician advances medical lead 160 through vasculature of patient 12 until electrode assemblies 170 (which may additionally or alternatively include one or more of electrode assembly 470, electrode assembly 770, electrode assembly 870, and/or electrode assembly 970 described in connection with FIG. 4B, FIGS. 7A and 7B, FIGS. 8A and 8B, FIGS. 9A and 9B respectively) are located within jugular vein 13 and positioned adjacent vagus nerve 21. In other examples, a clinician advances medical lead 160 through vasculature of patient 12 until electrode assemblies 170 are located within a cranial blood vessel proximate one or more target brain structures. Once electrode assemblies 170 are adjacent the target location (e.g., vagus nerve 21, other nerve, or one or more brain structures), the clinician initiates (e.g., via programmer 20, or another suitable device) electrical stimulation therapy and/or sensing of one or more patient parameters by medical device 14 via electrode assemblies 170.

In some examples, expandable structure 190, which can be at distal portion 150 of medical lead 160, is configured transform from a relatively low-profile delivery configuration to a deployed configuration in a blood vessel of a patient (e.g., within jugular vein 13 of patient 12). In some examples, expandable structure 190 remains in the delivery configuration during advancement of medical lead through the vasculature.

In some examples, expandable structure 190 transforms to the deployed (e.g., expanded) configuration once electrode assemblies 170 are adjacent the target site. In the deployed configuration of expandable structure 190, one or more of electrode assemblies 170 (e.g., one or more of electrode assembly 470, electrode assembly 770, electrode assembly 870, and/or electrode assembly 970) can be positioned into apposition with the vessel wall (e.g., the vessel wall of jugular vein 13). In some examples, electrode assemblies 170 (e.g., one or more of electrode assembly 470, electrode assembly 770, electrode assembly 870, and/or electrode assembly 970) are configured to bias transmissions of electrical signals to and/or from tissue surrounding the blood vessel (jugular vein 13) as compared to radially inward from the blood vessel wall (e.g., towards central longitudinal axis 191). For example, electrode assemblies 170 can be configured with an electrically insulative portion configured to face radially inward from expandable structure 190 (e.g., electrically insulative portion 880 as described with respect to FIG. 8A and FIG. 8B and/or electrically insulative portion 980 as described with respect to FIG. 9A and FIG. 9B). The biasing of transmissions of electrical signals to and/or from tissue surrounding the blood vessel can help facilitate directional electrical stimulation and/or sensing. In this way, the endovascular devices described herein can help target any suitable target tissue sites (e.g., nerve structures and/or brain structures) from an endovascular location and/or help avoid nontarget tissue sites, e.g., those associated with negative side effects.

The techniques described in this disclosure, including those attributed to medical device 14, programmer 20, or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as clinician or patient programmers, medical devices, or other devices. Processing circuitry, control circuitry, and sensing circuitry, as well as other processors and controllers described herein, may be implemented at least in part as, or include, one or more executable applications, application modules, libraries, classes, methods, objects, routines, subroutines, firmware, and/or embedded code, for example. In addition, analog circuits, components and circuit elements may be employed to construct one, some or all of the processing circuitry 30, instead of or in addition to the partially or wholly digital hardware and/or software described herein. Accordingly, analog or digital hardware may be employed, or a combination of the two.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. The computer-readable medium may be an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include RAM, ROM, programmable ROM (PROM), erasable programmable ROM (EPROM), electronically erasable programmable ROM (EEPROM), flash memory, a hard disk, a compact disc ROM (CD-ROM), a floppy disk, a cassette, magnetic media, optical media, or any other computer readable storage devices or tangible computer readable media.

In some examples, a computer-readable storage medium comprises non-transitory medium. The term “non-transitory” may indicate that the storage medium is not embodied in a carrier wave or a propagated signal. In certain examples, a non-transitory storage medium stores data that can, over time, change (e.g., in RAM or cache).

The functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements.

This disclosure includes the following non-limiting examples.

Example 1: An endovascular medical device system includes an elongated body configured to be introduced in a blood vessel of a patient; an expandable structure at a distal portion of the elongated body, the expandable structure includes an expandable body portion including a plurality of interconnected struts; and a plurality of electrode attachment elements, each electrode attachment element of the plurality of electrode attachment elements including one or more projections branching off of a strut of the plurality of interconnected struts; and one or more electrode assemblies mechanically coupled to the expandable structure via at least one electrode attachment element of the plurality of electrode attachment elements, each respective electrode assembly of the one or more electrode assemblies includes an electrically conductive portion; and an electrically insulative portion configured to receive the one or more projections, wherein each electrode attachment element of the plurality of electrode attachment elements is configured to orient a respective electrode assembly of the one or more electrode assemblies such that the electrically conductive portion faces radially outward from the expandable structure.

Example 2: The endovascular medical device system of example 1, wherein the expandable structure is configured to expand radially outwards from a relatively low-profile delivery configuration to a deployed configuration to position the one or more electrode assemblies to deliver electrical stimulation to tissue of the patient or sense a patient parameter from a location within the blood vessel.

Example 3: The endovascular medical device system of any of examples 1 or 2, wherein at least a subset of the plurality of electrode attachment elements are configured to orient respective electrode assemblies such that the electrically insulative portion faces radially inward from the expandable structure.

Example 4: The endovascular medical device system of any of examples 1 through 3, wherein the plurality of electrode attachment elements is configured to minimize rotation of a given electrode assembly of the one or more electrode assemblies around one or more struts of the plurality of interconnected struts.

Example 5: The endovascular medical device system of any of examples 1 through 4, wherein the electrically insulative portion of each electrode assembly defines one or more fixation holes configured to receive the one or more projections of a respective electrode attachment element.

Example 6: The endovascular medical device system of example 5, wherein at least one fixation hole of the one or more fixation holes is a through hole.

Example 7: The endovascular medical device system of example 5, wherein at least one fixation hole of the one or more fixation holes is a blind hole.

Example 8: The endovascular medical device system of example 5, wherein the one or more fixation holes includes at least a first fixation hole and a second fixation hole, wherein the one or more projections of the respective electrode attachment element includes at least a first projection and a second projection, wherein the first fixation hole is configured to receive the first projection and the second fixation hole is configured to receive the second projection.

Example 9: The endovascular medical device system of any of examples 1 through 8, wherein each electrode assembly of the one or more electrode assemblies defines at least one conductor hole configured to receive at least one conductor wire.

Example 10: The endovascular medical device system of example 9, wherein the at least one conductor hole is a through hole, and wherein the at least one conductor hole includes an electrical contact electrically connected to the electrically conductive portion of the respective electrode assembly.

Example 11: The endovascular medical device system of example 9, wherein the at least one conductor hole is a blind hole, and wherein the at least one conductor hole includes an electrical contact electrically connected to the electrically conductive portion of the respective electrode assembly.

Example 12: The endovascular medical device system of any of examples 9 through 11, wherein the at least one conductor hole includes a first conductor hole and a second conductor hole and the at least one conductor wire includes at least a first conductor wire and a second conductor wire, wherein the electrically insulative portion defines the second conductor hole, the second conductor hole configured to receive the second conductor wire, and wherein the second conductor hole configured as a pass-through hole to facilitate electrical connection of the second conductor wire to a second, different electrode assembly of the one or more electrode assemblies.

Example 13: The endovascular medical device system of any of examples 1 through 12, wherein the electrically insulative portion defines one or more slots, each slot of the one or more slots configured to receive a portion of the plurality of interconnected struts.

Example 14: The endovascular medical device system of example 13, wherein the one or more slots are configured to minimize rotation of a given electrode assembly of the one or more electrode assemblies around one or more struts of the plurality of interconnected struts.

Example 15: The endovascular medical device system of any of examples 13 or 14, wherein the one or more slots includes at least two slots, wherein each electrode assembly of the one or more electrode assemblies defines at least one conductor hole configured to receive a conductor wire, and wherein the at least one conductor hole is positioned between the at least two slots.

Example 16: The endovascular medical device system of any of examples 1 through 15, wherein each electrode assembly of the one or more electrode assemblies is configured to be secured to a respective electrode attachment element via an interference fit between the electrically insulative portion and the one or more projections of the respective electrode attachment element.

Example 17: The endovascular medical device system of example 16, wherein the one or more projections include one or more barbs, the one or more barbs configured to create the interference fit between each electrode assembly and the respective electrode attachment element.

Example 18: The endovascular medical device system of any of examples 1 through 17, wherein a portion of the one or more projections received by the respective electrode assembly is substantially parallel to the strut.

Example 19: The endovascular medical device system of any of examples 1 through 17, wherein a portion of the one or more projections received by the respective electrode assembly is not parallel to the strut.

Example 20: The endovascular medical device system of any of examples 1 through 19, wherein at least one electrode assembly of the one or more electrode assemblies is reflectionally symmetric about a plane intersecting a midpoint on an electrode assembly face.

Example 21: The endovascular medical device system of any of examples 1 through 20, wherein the electrically conductive portion includes a hollow cylinder, and wherein the electrically insulative portion includes a molded polymer configured to cover at least a portion of the hollow cylinder.

Example 22: A method of using a medical device system includes introducing a medical device into vasculature of a patient, the medical device includes an elongated body configured to be introduced in a blood vessel of a patient; an expandable structure at a distal portion of the elongated body, the expandable structure includes an expandable body portion including a plurality of interconnected struts; and a plurality of electrode attachment elements, each electrode attachment element of the plurality of electrode attachment elements including one or more projections branching off of a strut of the plurality of interconnected struts; and one or more electrode assemblies mechanically coupled to the expandable structure via at least one electrode attachment element of the plurality of electrode attachment elements, each respective electrode assembly of the one or more electrode assemblies includes an electrically conductive portion; and an electrically insulative portion configured to receive the one or more projections, wherein each electrode attachment element of the plurality of electrode attachment elements is configured to orient a respective electrode assembly of the one or more electrode assemblies such that the electrically conductive portion faces radially outward from the expandable structure; and advancing the medical device until the one or more electrode assemblies are at or near a target location in the vasculature of the patient.

Example 23: The method of example 22, wherein the expandable structure is configured to expand radially outwards from a relatively low-profile delivery configuration to a deployed configuration to position the one or more electrode assemblies to deliver electrical stimulation to tissue of the patient or sense a patient parameter from a location within the blood vessel.

Example 24: The method of any of examples 22 or 23, wherein at least a subset of the plurality of electrode attachment elements are configured to orient respective electrode assemblies such that the electrically insulative portion faces radially inward from the expandable structure.

Example 25: The method of any of examples 22 through 24, wherein the plurality of electrode attachment elements is configured to minimize rotation of a given electrode assembly of the one or more electrode assemblies around one or more struts of the plurality of interconnected struts.

Example 26: The method of any of examples 22 through 25, wherein the electrically insulative portion of each electrode assembly defines one or more fixation holes configured to receive the one or more projections of a respective electrode attachment element.

Example 27: The method of example 26, wherein at least one fixation hole of the one or more fixation holes is a through hole.

Example 28: The method of example 26, wherein at least one fixation hole of the one or more fixation holes is a blind hole.

Example 29: The method of example 26, wherein the one or more fixation holes includes at least a first fixation hole and a second fixation hole, wherein the one or more projections of the respective electrode attachment element includes at least a first projection and a second projection, wherein the first fixation hole is configured to receive the first projection and the second fixation hole is configured to receive the second projection.

Example 30: The method of any of examples 22 through 29, wherein each electrode assembly of the one or more electrode assemblies defines at least one conductor hole configured to receive at least one conductor wire.

Example 31: The method of example 30, wherein the at least one conductor hole is a through hole, and wherein the at least one conductor hole includes an electrical contact electrically connected to the electrically conductive portion of the respective electrode assembly.

Example 32: The method of example 30, wherein the at least one conductor hole is a blind hole, and wherein the at least one conductor hole includes an electrical contact electrically connected to the electrically conductive portion of the respective electrode assembly.

Example 33: The method of any of examples 30 through 32, wherein the at least one conductor hole includes a first conductor hole and a second conductor hole and the at least one conductor wire includes at least a first conductor wire and a second conductor wire, wherein the electrically insulative portion defines the second conductor hole, the second conductor hole configured to receive the second conductor wire, and wherein the second conductor hole configured as a pass-through hole to facilitate electrical connection of the second conductor wire to a second, different electrode assembly of the one or more electrode assemblies.

Example 34: The method of any of examples 22 through 33, wherein the electrically insulative portion defines one or more slots, each slot of the one or more slots configured to receive a portion of the plurality of interconnected struts.

Example 35: The method of example 34, wherein the one or more slots are configured to minimize rotation of a given electrode assembly of the one or more electrode assemblies around one or more struts of the plurality of interconnected struts.

Example 36: The method of any of examples 34 or 35, wherein the one or more slots includes at least two slots, wherein each electrode assembly of the one or more electrode assemblies defines at least one conductor hole configured to receive a conductor wire, and wherein the at least one conductor hole is positioned between the at least two slots.

Example 37: The method of any of examples 22 through 36, wherein each electrode assembly of the one or more electrode assemblies is configured to be secured to a respective electrode attachment element via an interference fit between the electrically insulative portion and the one or more projections of the respective electrode attachment element.

Example 38: The method of example 37, wherein the one or more projections include one or more barbs, the one or more barbs configurated to create the interference fit between each electrode assembly and the respective electrode attachment element.

Example 39: The method of any of examples 22 through 38, wherein a portion of the one or more projections received by the respective electrode assembly is substantially parallel to the strut.

Example 40: The method of any of examples 22 through 39, wherein a portion of the one or more projections received by the respective electrode assembly is not parallel to the strut.

Example 41: The method of any of examples 22 through 40, wherein at least one electrode assembly of the one or more electrode assemblies is reflectionally symmetric about a plane intersecting a midpoint on an electrode assembly face.

Example 42: The method of any of examples 22 through 41, wherein the electrically conductive portion includes a hollow cylinder, and wherein the electrically insulative portion includes a molded polymer configured to cover at least a portion of the hollow cylinder.

Example 43: An electrode assembly includes an electrically conductive portion configured to transmit electrical signals, and an electrically insulative portion, the electrically insulative portion molded around a surface of the electrically conductive portion, wherein one or more of the electrically conductive portion and the electrically insulative portion defines a conductor hole configured to receive a conductor wire, and wherein the electrically insulative portion defines at least one fixation hole configured to receive a projection of an expandable structure to mechanically couple the electrode assembly to the expandable structure.

Example 44: The electrode assembly of example 43, wherein the conductor hole is a first conductor hole and the conductor wire is a first conductor wire, and wherein the electrically insulative portion defines a second conductor hole configured to receive a second conductor wire, the second conductor hole configured as a pass-through hole to facilitate electrical connection of the second conductor wire to a second, different electrode assembly.

Various examples of the disclosure have been described. Any combination of the described systems, operations, or functions is contemplated. These and other examples are within the scope of the following claims.

What is claimed is:

1. An endovascular medical device system comprising:
an elongated body configured to be introduced in a blood vessel of a patient;
an expandable structure at a distal portion of the elongated body, the expandable structure comprising:
an expandable body portion including a plurality of interconnected struts; and
a plurality of electrode attachment elements, each electrode attachment element of the plurality of electrode attachment elements including one or more projections branching off of a strut of the plurality of interconnected struts; and
one or more electrode assemblies mechanically coupled to the expandable structure via at least one electrode attachment element of the plurality of electrode attachment elements, each respective electrode assembly of the one or more electrode assemblies comprising:
an electrically conductive portion; and
an electrically insulative portion configured to receive the one or more projections,
wherein each electrode attachment element of the plurality of electrode attachment elements is configured to orient a respective electrode assembly of the one or more electrode assemblies such that the electrically conductive portion faces radially outward from the expandable structure.

2. The endovascular medical device system of claim 1, wherein the expandable structure is configured to expand radially outwards from a relatively low-profile delivery configuration to a deployed configuration to position the one or more electrode assemblies to deliver electrical stimulation to tissue of the patient or sense a patient parameter from a location within the blood vessel.

3. The endovascular medical device system of claim 2, wherein at least a subset of the plurality of electrode attachment elements are configured to orient respective electrode assemblies such that the electrically insulative portion faces radially inward from the expandable structure.

4. The endovascular medical device system of claim 3, wherein the plurality of electrode attachment elements is configured to minimize rotation of a given electrode assembly of the one or more electrode assemblies around one or more struts of the plurality of interconnected struts.

5. The endovascular medical device system of claim 4, wherein the electrically insulative portion of each electrode assembly defines one or more fixation holes configured to receive the one or more projections of a respective electrode attachment element.

6. The endovascular medical device system of claim 5, wherein at least one fixation hole of the one or more fixation holes is a through hole.

7. The endovascular medical device system of claim 5, wherein at least one fixation hole of the one or more fixation holes is a blind hole.

8. The endovascular medical device system of claim 5,
wherein the one or more fixation holes includes at least a first fixation hole and a second fixation hole,
wherein the one or more projections of the respective electrode attachment element includes at least a first projection and a second projection, wherein the first fixation hole is configured to receive the first projection and the second fixation hole is configured to receive the second projection.

9. The endovascular medical device system of claim 8, wherein each electrode assembly of the one or more electrode assemblies defines at least one conductor hole configured to receive at least one conductor wire, and wherein the at least one conductor hole is a through hole, and wherein the at least one conductor hole includes an electrical contact electrically connected to the electrically conductive portion of the respective electrode assembly.

10. The endovascular medical device system of claim 9, wherein the at least one conductor hole is a blind hole, and wherein the at least one conductor hole includes an electrical contact electrically connected to the electrically conductive portion of the respective electrode assembly.

11. The endovascular medical device system of claim 9,
wherein the at least one conductor hole includes a first conductor hole and a second conductor hole and the at least one conductor wire includes at least a first conductor wire and a second conductor wire,
wherein the electrically insulative portion defines the second conductor hole, the second conductor hole configured to receive the second conductor wire, and
wherein the second conductor hole configured as a pass-through hole to facilitate electrical connection of the second conductor wire to a second, different electrode assembly of the one or more electrode assemblies.

12. The endovascular medical device system of claim 1, wherein the electrically insulative portion defines one or more slots, each slot of the one or more slots configured to receive a portion of the plurality of interconnected struts.

13. The endovascular medical device system of claim 12, wherein the one or more slots are configured to minimize rotation of a given electrode assembly of the one or more electrode assemblies around one or more struts of the plurality of interconnected struts.

14. The endovascular medical device system of claim 12,
wherein the one or more slots includes at least two slots,
wherein each electrode assembly of the one or more electrode assemblies defines at least one conductor hole configured to receive a conductor wire, and
wherein the at least one conductor hole is positioned between the at least two slots.

15. The endovascular medical device system of claim 1, wherein each electrode assembly of the one or more electrode assemblies is configured to be secured to a respective electrode attachment element via an interference fit between the electrically insulative portion and the one or more projections of the respective electrode attachment element.

16. The endovascular medical device system of claim 15, wherein the one or more projections include one or more barbs, the one or more barbs configured to create the interference fit between each electrode assembly and the respective electrode attachment element.

17. The endovascular medical device system of claim 1, wherein a portion of the one or more projections received by the respective electrode assembly is substantially parallel to the strut.

18. The endovascular medical device system of any of claim 1, wherein a portion of the one or more projections received by the respective electrode assembly is not parallel to the strut.

19. The endovascular medical device system of claim 1, wherein at least one electrode assembly of the one or more electrode assemblies is reflectionally symmetric about a plane intersecting a midpoint on an electrode assembly face.

20. The endovascular medical device system of claim 1, wherein the electrically conductive portion includes a hollow cylinder, and wherein the electrically insulative portion includes a molded polymer configured to cover at least a portion of the hollow cylinder.
